(19) United States
(12) Patent Application Publication
(10) Pub. No.: US 2020/0215228 A1
(43) Pub. Date: Jul. 9, 2020

(54) COLLAGEN MICROFIBER SCAFFOLDS

(71) Applicant: BROWN UNIVERSITY, Providence, RI (US)

(72) Inventors: Kareen L. K. COULOMBE, Pawtucket, RI (US); Nicholas J. KAISER, Providence, RI (US); Jessica Anna BELLOWS, Hope, RI (US)

(21) Appl. No.: 16/723,579

(22) Filed: Dec. 20, 2019

Related U.S. Application Data
(60) Provisional application No. 62/783,030, filed on Dec. 20, 2018.
(60) Provisional application No. 62/783,456, filed on Dec. 21, 2018.

(51) Int. Cl. A61L 27/24; A61L 27/48; A61L 27/52; D01D 5/00

(52) U.S. Cl. CPC A61L 27/24; D01D 5/0046; A61L 27/52; A61L 27/48

(57) ABSTRACT

The invention features a system for preparing composite collagen microfiber scaffolds and biomaterials with a broad range of mechanical properties and uses. Using low cost materials, rapid fabrication techniques, and accessible software tools, the system provides a customizable, automated, biomaterial fabrication platform with broad accessibility.

### CROSS-REFERENCE TO RELATED APPLICATIONS

This patent application claims priority under 35 U.S.C. § 119(e) to the provisional patent applications U.S. Ser. No. 62/783,030, filed Dec. 20, 2018, and U.S. Ser. No. 62/783,456, filed Dec. 21, 2018.

### FIELD OF THE INVENTION

This invention generally relates to a replacement or repair of human tissue, and particularly to collagen microfiber scaffolds. This invention also relates to the mechanical treatment of natural fibrous or filamentary material to obtain fibers or filaments.

### BACKGROUND OF THE INVENTION

Injury and trauma can lead to damage and degeneration of tissues in the human body, which necessitates treatments to facilitate their repair, replacement, or regeneration. Treatment of damage and degeneration of tissues often involves transplanting tissue from one site to another in the same patient (an autograft) or from one individual to another (a transplant or allograft).

Harvesting autografts remains expensive, painful, constrained by anatomical limitations, and associated with donor-site morbidity because of infection and hematoma. Allografts and transplants have severe constraints because of the difficulties with accessing enough tissue for all the patients who require them. Allografts and transplants also carry risks of rejection by the patients' immune systems and risks of introducing infection or disease from the donor to the patient.

By contrast, the field of tissue engineering aims to regenerate damaged tissues, instead of replacing them, by developing biological substitutes that restore, maintain, or improve tissue function.

There remains a need in the tissue engineering art to regenerate damaged tissues by combining cells from the body with highly porous scaffold biomaterials, which act as templates for tissue regeneration, to guide the growth of new tissue.

### SUMMARY OF THE INVENTION

The invention features a system for preparing collagen microfiber scaffolds and biomaterials with a broad range of mechanical properties and uses. Using low cost materials, rapid fabrication techniques, and accessible software tools, the system provides a customizable, automated, biomaterial fabrication platform with broad accessibility.

In the first embodiment, the invention provides a composite collagen microfiber scaffold. The scaffold is formed from wet spun collagen microfibers by coaxial flow by a buffering reaction. The collagen microfiber has native collagen molecular structure. The reaction provides control of microfiber diameter.

In the second embodiment, the invention provides a high viscosity wet spinning buffer composition (HV-WSB) In a specific embodiment, the buffer composition is 34.5 mM potassium phosphate monobasic, 85.2 mM sodium phosphate dibasic, 135 mM sodium chloride, 29.9 mM HEPES buffer, 8.57 mM polyethylene glycol (PEG) MW 35,000.

In the third embodiment, the system further provides control of microfiber spacing, angles, and layering.

In the fourth embodiment, the invention provides a mesh collection and organization device with multiple scaffold windows for maintaining fiber cross-sectional shape.

In the fifth embodiment, the invention provides a collagen fiber wet spinning and mesh device with coaxial fiber formation, washing, fiber guidance onto the collection device, and independent rotational and translational control for mesh collection. Features of the coaxial fiber formation spinneret elements are shown in FIG. 1A and FIG. 1B. Washing is done, e.g., in an ethanol bath. See FIG. 1C.

In the sixth embodiment, the invention provides a collagen microfiber mesh with control of microfiber spacing, angles, and layering. With 400 μm spacing, effective dry fiber diameters were consistent (51.26±11.16 μm, mean±SD). This variability also increased when spacing became small because of electrostatic interactions that drew adjacent fibers together (81.85±80.38 for 100 μm, mean±SD). The device presented in this specification can produce meshes with fiber spacing about 100 μm.

In the seventh embodiment, the invention provides a facile, low-cost, and automated method of preparing collagen microfibers. The steps of the method are organizing the collagen fibers into precisely controlled mesh designs, embedding the mesh design of collagen fibers in a bulk hydrogel, and creating a biomaterial suitable for a wide variety of tissue engineering and regenerative medicine applications. The resulting collagen microfibers demonstrate high precision and reproducibility in both fiber and mesh fabrication. When evaluated for their single fiber mechanical properties, these collagen microfibers are evidence of collagen self-assembly in the microfibers under standard cell culture conditions. The physical and biological characterizations of these collagen microfiber meshes demonstrate physiologically relevant mechanical properties, native-like collagen structure, and cytocompatibility.

In the eighth embodiment, the invention provides an automated method for the fabrication of bespoke collagen microfiber scaffolds for tissue. The method comprises the steps of isolating fibrillary collagen; fabricating and assembling a collection device and bath; designing microfiber mesh protocols; wet spinning collagen meshes; and capturing and embedding the wet spun collagen meshes. In the ninth embodiment, the fibrillary collagen is a vertebrate type I collagen. In the tenth embodiment, the fibrillary collagen is isolated from human cadaver tendon, bovine skin, or rat tail tendon.

In the eleventh embodiment, the invention provides a method comprising the steps of preparing collagen microfibers, organizing the microfibers into precisely controlled mesh designs, and embedding the tissues in a bulk hydrogel. In the twelfth embodiment, the method further comprises calibration and translation from digital design to physical mesh for precise control. In the thirteenth embodiment, the method further comprises creating a platform suitable for a wide variety of tissue engineering applications.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 is a set of drawings illustrating a collagen fiber wet spinning and mesh device. FIG. 1(A) is a cross-sectional schematic of the spinneret composed of a 22-gauge syringe needle (red) inserted into the needle cap. FIG. 1(B) is a photograph of the spinneret. Collagen enters the spinneret through syringe pump extrusion and forms a co-axial flow system with wet-spin buffer descending from the buffer reservoir. Windows are cut in the needle cap to allow the buffer to flow in from the sides. The end of the cap was cut to connect with the exit tubing. FIG. 1(C) is an overhead photograph of the bath and collector. The continuous collagen fiber exits into the bath filled with 70% ethanol, which washes away residual polyethylene glycol (PEG) and facilitates fiber drying. The fiber is pulled through the length of the bath, threaded through a fiber guide, and laid onto the mandrel of the collection device. FIG. 1(D) is a close-up photograph of a continuous collagen fiber traveling through the 70% ethanol bath. The white dotted line box indicates the location of the continuous collagen microfiber. FIG. 1(E) is a close-up photograph of the fiber guide and the collection mandrel. FIG. 1(F) is a close-up photograph of the fiber collector, which includes a fiber guide and the mandrel on a translating platform. Two stepper motors, driven by an Arduino microcontroller, direct the rotation and translation of the mandrel. FIG. 1(G) is a photograph of an Arduino microcontroller that controls the collection device and executes mesh protocols. FIG. 1(H) is a photograph of a 30° collagen microfiber mesh on the mandrel.

FIG. 2 is a set of drawings illustrating a collagen mesh capture frame. FIG. 2(A) is an exploded schematic of the silicone gaskets and steel support frames placed on opposite sides of a collagen microfiber mesh on the collection mandrel. The frames are then collapsed together, and the screws are put in place to hold the frame together. FIG. 2(B) is a photograph of the screws, frames, and gaskets used in the capture process. FIG. 2(C) is a photograph of a captured mesh in the well of a six-well plate, ready for hydrogel/cell casting.

FIG. 3 is a set of drawings illustrating a mechanical analysis of wet spun collagen microfibers. FIG. 3(A) is a photograph showing the micromechanics setup, which includes one hook connected to a five mN load cell and one hook attached to a lever arm. FIG. 3(B) is a photograph showing individual collagen microfibers glued to stainless steel washers before incubation in phosphate-buffered saline (PBS) at 37° C. FIG. 3(C) is a photograph showing a hydrated collagen fiber sample mounted on the mechanics setup before analysis. FIGS. 3(D-F) is a set of bar graphs showing Young's modulus, ultimate tensile stress (UTS), and strain at break for individual collagen microfibers incubated for 0-96 hours. FIG. 3(G) is a set of bar graphs showing Young's modulus of individual collagen microfibers crosslinked by heat (one hour), ultraviolet (UV) (one hour), or glutaraldehyde vapor (twenty-four hours) before incubation. *p<0.05, **p<0.001, and ***p<0.0001.

FIG. 4 is a set of drawings illustrating the evaluation of wet spun mesh fidelity. FIG. 4(A) is a set of digital design renderings for parallel fiber meshes with 100 μm, 200 μm, and 400 μm fiber spacing (top) and light microscopy images of physical meshes prepared using these protocols (bottom). FIG. 4(B) is a bar graph showing a quantitative analysis of parallel fiber fidelity based on diameter. FIG. 4(C) is a bar graph showing gap measurements of the 100 μm, 200 μm, and 400 μm spaced parallel fiber meshes by image analysis software. Error bars represent standard deviation (SD). FIG. 4(D) is a digital design rendering of a 30°, 200 μm fiber spacing mesh. FIG. 4(E) is a gross image of wet-spun 30°, 200 μm fiber spacing mesh with image analysis fiber angle overlay. FIG. 4(F) is a graph showing mesh angles produced from the 30°, 200 μm fiber spacing protocol before and after software calibration. FIG. 4(G) is a graph showing mesh angles produced in each of four meshes from three separate fabrication batches using the 30°, 200 μm fiber spacing protocol.

FIG. 5 is a set of drawings illustrating an exemplary bidirectional mechanical analysis. FIG. 5(A) is a diagram of 30° fiber angle mesh captured in the transverse (left) and longitudinal (right) orientations. FIG. 5(B) is a photograph of a longitudinal 30° fiber angle mesh during mechanical testing. FIG. 5(C) is a bar graph showing Young's modulus values for 30° and 60° fiber angle mesh evaluated in both transverse and longitudinal directions. *p<0.05, **p<0.001.

FIG. 6 is a set of schematics showing laser cut acrylic parts for fiber collector and mandrel. These schematics are for reference only. Persons of ordinary skill in the tissue engineering art when laser cutting can use the freely available.ai files provided by Kaiser et al., Tissue Eng. Part C Methods, 25(11), 687-700. (Nov. 1, 2019), “Supplementary Table S1,” at: http://www.liebertpub.com/doi/full/10.1089/ten.TEC.2018.0379?url_ver=Z39.88-2003&fr_id=ori:rid:crossreforg&rfr_dat=cr_pub %3dpubmed.

FIG. 7 is a set of photographs showing reference images for the fiber collector and mandrel. The letters in FIG. 7 refer to the corresponding elements in FIG. 6.

FIG. 8 is a set of schematics showing laser cut acrylic parts for wet spinning bath. These schematics are for reference only. Persons of ordinary skill in the tissue engineering art when laser cutting can use the freely available.ai files provided by Kaiser et al., Tissue Eng. Part C Methods, 25(11), 687-700. (Nov. 1, 2019), “Supplementary Table S1,” at: https://www.liebertpub.com/doi/full/10.1089/ten.TEC.2018.0379?url_ver=Z39.88-2003&rfr_id=ori:rid:crossref.org&rfr_dat=cr_pub%3dpubmed.

FIG. 9 is a set of schematics showing laser cut acrylic parts for an alignment jig. These schematics are for reference only. Persons of ordinary skill in the tissue engineering art when laser cutting can use the freely available.ai files provided by Kaiser et al., Tissue Eng. Part C Methods, 25(11), 687-700. (Nov. 1, 2019), “Supplementary Table S1,” at: https://www.liebertpub.com/doi/full/10.1089/ten.TEC.2018.0379?url_ver=Z39.88-2003&rfr_id=ori:rid:crossref.org&rfr_dat=cr_pub %3dpubmed.

FIG. 10 is a pair of photographs showing a reference image for an assembled alignment jig in the open and closed configurations.

FIG. 11 is a set of schematics showing design plans for machined steel parts for the fiber collector.

FIG. 12 is a set of schematics showing design plans for steel frames and silicone gaskets described in FIG. 2.

FIG. 13 shows mesh patterns designed via a digital graphical user interface (GUI; top row) and translated into protocols executed by a custom mesh collection and organization device (bottom row).

FIG. 14A shows an exemplary python code for. dia to .ino translation.

FIG. 14B shows an exemplary python code for. dia to .ino translation.

FIG. 14C shows an exemplary python code for. dia to .ino translation.

FIG. 14D shows an exemplary python code for. dia to .ino translation.

FIG. 14E shows an exemplary python code for. dia to .ino translation.

FIG. 14F shows an exemplary python code for. dia to .ino translation.

FIG. 14G shows an exemplary python code for. dia to .ino translation.

FIG. 14H shows an exemplary python code for. dia to .ino translation.

### DETAILED DESCRIPTION OF THE INVENTION

State of the Art

Many natural and synthetic polymer materials have been used in soft tissue engineering as extracellular matrix materials. Collagen and fibrin hydrogels have been widely used because of their ready availability, ease of use, ability to be remodeled by resident and host cells, minimal immune response, and high density of cell adhesion sites compared to their synthetic polymer counterparts. Collagen and fibrin hydrogels have been widely used in humans. However, these and other hydrogels lack the structural and mechanical anisotropy that characterize the extracellular matrix in many tissues, such as skeletal and cardiac muscle, tendon, and cartilage.

Persons of ordinary skill in the tissue engineering art often use stress fields resulting from tissue compaction in isometrically confined tissues as a surrogate to induce cell alignment. While often effective at inducing alignment, the available methods necessitate either tissue fenestrations (reducing the efficacy and efficiency of tissues with function related to mechanics and structure) or prescribe high-aspect-ratio tissues with limited utility as a replacement tissue patches. This approach does not replicate the mechanical material anisotropy found in these native tissues and extracellular matrix (ECM), which is often a defining feature of these tissues and materials, and which may be important in cell and tissue development.

The soft tissue engineering community has used a wide variety of natural and synthetic polymer hydrogel materials as accessible scaffold materials, permitting high customization for individual applications, as reviewed by Jafari et al., J. Biomed. Mater. Res. B Appl. Biomater. 105, 431 (2017). Tissue engineers can select from a vast library of hydrogel scaffolds defined by molecular composition and organization. Tissue engineers can therefore choose a composition best suited to their cell and tissue type of interest in terms of cell adhesion site availability and density, mechanical stiffness and strength, remodeling and degradation rates, cleavage sites, and many other parameters for tissue development. However, these systems have a limited ability to provide internal structural and organizational cues to resident cells.

Polymer microfibers embedded in a bulk hydrogel provide a way of providing precise structural and mechanical cues to cells seeded in engineered tissues, offering tissue engineers another dimension of control in designing tissues for therapy and research. Several strategies to emulating extracellular matrix structural and mechanical cues in 3D engineered tissues in an array of shapes have been used in the tissue engineering art, including aligned and unorganized nanofiber mats, aligned pores through directional freezing, sphere- and rod-templated scaffolds, and 3D printed scaffolds. All these strategies have effected significant changes on cell phenotype and function, thereby demonstrating their value to the field and the importance of these mechanical and structural signaling cues. However, all these strategies require compromise in terms of thickness limitations, ease of cell infiltration, and precise control over the scaffold pattern or morphology. Aligned and unorganized nanofiber mats feature high surface areas and fiber densities, but the high fiber density can make cell infiltration challenging, especially in thicker mats. The fabrication of complex, well-defined patterns remain a challenge, despite recent innovations in the collection of aligned electrospun fiber arrays. Directional freezing can create uniform, anisotropic pore arrays in a variety of natural and synthetic scaffold materials, but directional controlled rate freezing has constraints, such as singular freezing direction, which limit the types of morphologies that can be created. 3D printed scaffolds offer high reproducibility and a broad design space, but compromises must be made in terms of either resolution or scaffold polymer, often resulting in excluding natural polymer scaffolds.

An alternative, bioinspired solution uses polymer microfibers embedded in a bulk hydrogel, which presents a way of providing precise structural and mechanical cues to cells seeded in engineered tissues and provides tissue engineers another dimension of control in designing tissues for specific applications. Aligned arrays of microfibers composed of collagen, fibrin, and other natural polymers have been used as scaffolds in a variety of tissue systems and biomaterials with broader applications, such as wound dressings and sutures. Overlapping sets of natural polymer microfibers have also been used to create more sophisticated fibrous tissue scaffolds and therapeutic biomaterials with unique and tunable properties. However, fabrication of natural polymer fibrous networks is more complex than overlapping aligned fibers. The broader adoption of these scaffolds and biomaterials has been hindered by technical challenges related to the characteristic fragility of these natural polymers, leading to processes that require significant manual labor at bench scale. Without automation, fabrication is constrained to simple patterns, low throughput, and low fiber density.

Innovation.

The inventors now present a facile, low-cost, and automated method of preparing collagen microfibers, organizing these fibers into precisely controlled mesh designs, and embedding these collagen microfiber tissues in a bulk hydrogel. Thus, the invention provides a method for creating a biomaterial suitable for a wide variety of tissue engineering and regenerative medicine uses.

The inventors successfully created a system for preparing composite collagen microfiber scaffolds and biomaterials with a broad range of mechanical properties and applications. The system is a customizable, automated, biomaterial fabrication platform with equally broad accessibility.

Using software tools such as those described in this specification, mesh patterns are designed using a digital graphical user interface (GUI) (see, FIG. 13) and translated into protocols executed by a custom mesh collection and organization device. The specification not only describes a process through which custom collagen microfiber meshes can be fabricated but also provide persons of ordinary skill in the tissue engineering art with detailed device plans and software tools to produce bespoke meshes through a precise, consistent, and automated process.

This specification presents and characterizes an automated method for the fabrication of high-fidelity, high density, collagen microfiber meshes. By using developed software tools, mesh patterns can be designed using a graphical user interface (GUI) and translated into automated fabrication protocols like those used by 3D printers, enabling the facile fabrication of complex designs. The specification also describes an aseptic method of capturing these meshes and embedding them in polymer hydrogels (such as natural collagen) as acellular biomaterials or with cells as engineered tissues. To ensure accessibility, this specification used low-cost materials and off-the-shelf components frequently for developing all associated tools and pieces of equipment.

The distinct advantages of incorporating wet-spun collagen microfibers into scaffolds presented are that it offers precise, customizable structural control and physiologically relevant mechanical robustness with no compromise in terms of cell adhesion site density, remodeling response, and immune response by simply using unadulterated collagen in both a structurally reinforcing fiber compartment and bulk hydrogel. These benefits are amplified by the low barrier to entry required for the automated approach provided in this specification.

### INDUSTRIAL APPLICABILITY

The collagen microfiber meshes of the invention have immediate and broad applications in tissue engineering. The collagen microfiber meshes have high potential for clinical therapeutics, because of their compositional similarities to native extracellular matrix and tunable structural and mechanical characteristics.

The methods in this specification not only describe a method by which custom collagen microfiber meshes can be fabricated but also provide persons of ordinary skill in the tissue engineering art with detailed device plans and software tools to produce their own bespoke meshes through a precise, consistent, and automated process.

This invention also provides a powerful, flexible platform for researchers studying tissue engineering and cell material interactions. This invention facilitates the development of therapeutic biomaterials in the form of custom collagen microfiber patterns accessible to throughout the methods and techniques described in this specification.

The versatile methods described in this specification provide a strong platform for direct application in both tissue engineering and biomaterial research, and to support further innovation in automated fabrication of customizable collagen materials.

Collagen microfiber meshes are a desirable platform for tissue engineering and biomaterial research because of the ability to tune both the mechanical properties of individual fibers and the composite as a whole with type 1 collagen, an inherently excellent cell adhesion and remodeling properties.

Adoption of bespoke collagen microfiber scaffolds benefit biomaterial engineering and fulfill a need for natural fibrous materials with defined architecture.

The recently understood role of a fibrous extracellular matrix microenvironment in cell and tissue function has increased interest in developing more sophisticated scaffold materials that can better emulate the diverse properties of native, fibrous extracellular matrix. As the field of tissue engineering matures and the translation and manufacturing of tissue therapies are developed, automated fabrication systems with high accuracy and precision offer substantial benefits over manual fabrication and assembly methods. Even at the bench scale, improvements to reproducibility and time costs are valuable. However, the typically high cost and complexity of automated fabrication for tissue engineering has limited the reach of these technologies in research settings. Minimal modification are needed to adapt the process and device described for mesh fabrication in a biosafety cabinet to eliminate bioburden concerns.

Automated fabrication systems with high accuracy and precision offer substantial benefits over manual fabrication and assembly methods for both research and therapeutic applications. Even at the bench scale, improvements to reproducibility and time costs are valuable. However, the typically high cost and complexity of automated fabrication for tissue engineering has limited the reach of these technologies in research settings. The simple method of mesh design and automated fabrication presented extends the advantages of automated biomaterial fabrication to a wide variety of applications and to users with any level of expertise to increase adoption of the collagen wet spinning method. Unlike electrospinning methods, collagen wet spinning is minimally affected by environmental parameters such as temperature, humidity, and airflow. Collagen wet spinning can be performed much more inexpensively and easily on an open benchtop or in a sterile environment such as a biosafety cabinet.

Embedded collagen microfibers provide a flexible platform for research because of the availability and relatively low cost of isolation from source tissues, besides the high density of cell adhesion sites, robust mechanics, native molecular structure, and tunable mechanical and degradation properties.

### Definitions

For convenience, the meaning of some terms and phrases used in the specification, examples, and appended claims, are listed below. Unless stated otherwise or implicit from context, these terms and phrases have the meanings. These definitions are to aid in describing embodiments and are not intended to limit the claimed invention. Unless otherwise defined, all technical and scientific terms have the same meaning as commonly understood by persons of ordinary skill in the tissue engineering art. For any apparent discrepancy between the meaning of a term in the art and a definition provided in this specification, the meaning provided in this specification shall prevail.

“About” has the plain meaning of approximately. The term “about” encompasses the measurement errors inherently associated with the relevant testing.

When used with percentages, “about” means ±1%. “About” or “approximately” when referring to a value or parameter means to be within a range of normal tolerance in the art, e.g., within two standard deviations of the mean. A description referring to “about X” includes description of “X.”

“Composite” scaffolds are scaffolds made up of more than one constituent, often one an organic material and another inorganic material that often have different physical forms in the combined scaffold, such as a fiber and a hydrogel.

“Fiber” has the has the weaving, knitting, and fiber art-recognized meaning of a natural or synthetic substance significantly longer than it is wide. Fibers are often used in manufacturing other materials. Other words that have the same meaning as fiber include thread, strand, tendril, filament, and fibril.

“Fibrillary collagens” or “fibrillar collagens (types I, II, III, V, XI, XXIV and XXVII) constitute a sub-group within the collagen family (of which there are 28 types in humans) whose functions provide three-dimensional frameworks for tissues and organs and assemble into fibrils and hierarchical fiber structures. These networks confer mechanical strength and signaling and organizing functions through binding to cellular receptors and other components of the extracellular matrix (ECM). See, Bella & Hulmes, Subcell. Biochem., 82, 457-490 (2017).

“Human induced pluripotent stem cells” or “hiPSC” are a type of pluripotent stem cell that can be generated directly from adult cells. Human induced pluripotent stem cells are a renewable source of human cells.

“Mesh design” is the practice of creating a mesh, a subdivision of a continuous geometric space into discrete geometric and topological units.

“Microfiber” is a fiber with a diameter in the micron length scale. The materials, methods and devices described in this specification can also produce larger (mm) fibers or smaller (nm) fibers. The inventive properties of the fibers and mesh of this invention encompasses microfibers, larger (mm) fibers, and smaller (nm) fibers

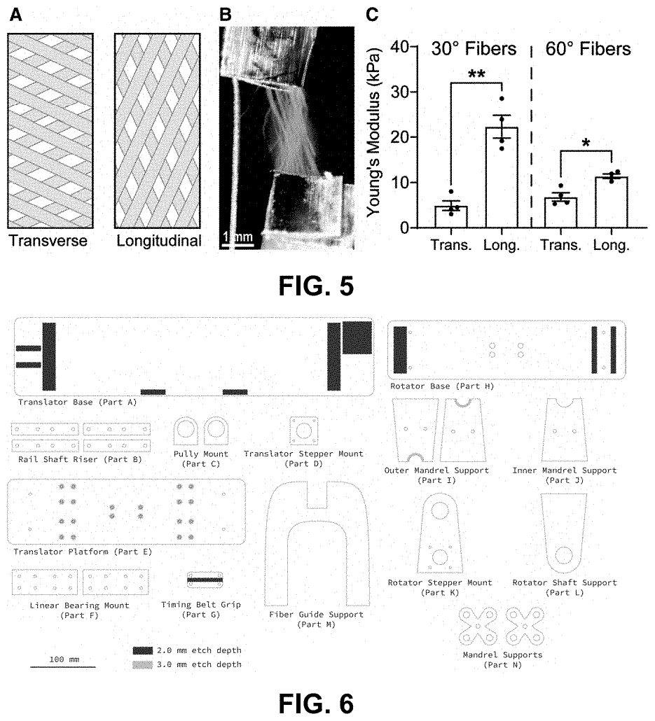

“Microfiber angle” has the weaving, knitting, and fiber art-recognized meaning of the angles between fibers. See, e.g., FIG. 4, FIG. 5, and FIG. 13.

“Microfiber diameter” has the weaving, knitting, and fiber art-recognized meaning of the diameter of a microfiber. The diameter of a fiber is a common measurement in the fiber art.

“Microfiber layering” has the weaving, knitting, and fiber art-recognized meaning of layering of fibers in the making of composite fibers.

“Microfiber spacing” has the weaving, knitting, and fiber art-recognized meaning of spacing between fibers, such as parallel fibers.

“Tissue engineering” is the use of a combination of cells, engineering, and materials methods, and suitable biochemical and physicochemical factors to improve, mimic, or replace biological tissues. Tissue engineering involves the use of a tissue scaffold for the formation of new viable tissue for a biological or medical purpose. The phrase “tissue engineering” is interchangeably used with “regenerative medicine.”

“Type I collagen” is the most abundant collagen of the human body. It forms large, eosinophilic fibers known as collagen fibers. It is present in scar tissue, the end product when tissue heals by repair, and tendons, ligaments, the endomysium of myofibrils, the organic part of bone, the dermis, the dentin, and organ capsules. The COL1A1 gene produces the pro-alpha1(I) chain. This chain combines with another pro-alpha1(I) chain and also with a pro-alpha2(I) chain (produced by the COL1A2 gene) to make a molecule of type I procollagen. These triple-stranded, rope-like procollagen molecules is processed by enzymes outside the cell. After these molecules are processed, they arrange themselves into long, thin fibrils that cross-link to one another in the spaces around cells. The cross-links result in the formation of very strong mature type I collagen fibers.

Guidance from the Prior Art

A person of ordinary skill in the tissue engineering art can use these scientific references as guidance to predictable results when making and using the invention.

Jafari et al., J. Biomed. Mater. Res. B Appl. Biomater., 105, 431 (2017) is a literature review of polymeric scaffolds in tissue engineering. The soft tissue engineering community has used a wide variety of natural and synthetic polymer hydrogel materials as accessible scaffold materials, permitting high customization for individual applications.

Bian & Bursac, Biofabrication, 6, 024109 (2014) describes the structural and functional anisotropy of engineered cardiac tissues. Stress fields resulting from tissue compaction in isometrically confined tissues are often used as a surrogate to induce cell alignment. Available methods necessitate either tissue fenestrations (reducing the efficacy and efficiency of tissues with function related to mechanics and structure) or prescribe high-aspect-ratio tissues with limited utility as a replacement tissue patches.

Munarin et al., Tissue Eng. Part C Methods 23, 311 (2017) provides laser-etched designs for molding hydrogel-based engineered tissues. Stress fields resulting from tissue compaction in isometrically confined tissues are often used as a surrogate to induce cell alignment. Available methods necessitate either tissue fenestrations (reducing the efficacy and efficiency of tissues with function related to mechanics and structure) or prescribe high-aspect-ratio tissues with limited utility as a replacement tissue patches.

Engelmayr et al., “Accordion-like honeycombs for tissue engineering of cardiac anisotropy.” Nat Mater 7, 1003, 2008. Stress fields resulting from tissue compaction in isometrically confined tissues are often used as a surrogate to induce cell alignment. Available methods necessitate either tissue fenestrations (reducing the efficacy and efficiency of tissues with function related to mechanics and structure) or prescribe high-aspect-ratio tissues with limited utility as a replacement tissue patches.

Zimmermann et al. “Engineered heart tissue grafts improve systolic and diastolic function in infarcted rat hearts.” Nat Med 12, 452, 2006. Stress fields resulting from tissue compaction in isometrically confined tissues are often used as a surrogate to induce cell alignment. Available methods necessitate either tissue fenestrations (reducing the efficacy and efficiency of tissues with function related to mechanics and structure) or prescribe high-aspect-ratio tissues with limited utility as a replacement tissue patches.

Sugimura & Ishihara, “The mechanical anisotropy in a tissue promotes ordering in hexagonal cell packing.” Dev. Camb. Engl. 140, 4091 (2013). Stress fields resulting from tissue compaction in isometrically confined tissues are often used as a surrogate to induce cell alignment. This approach does not replicate the mechanical material anisotropy found in these native tissues and extracellular matrix.

Place et al., “Complexity in biomaterials for tissue engineering.” Nat Mater 8, 457, 2009. Stress fields resulting from tissue compaction in isometrically confined tissues are often used as a surrogate to induce cell alignment. This approach does not replicate the mechanical material anisotropy found in these native tissues and extracellular matrix.

Jeffries et al., “Highly elastic and suturable electrospun poly(glycerol sebacate) fibrous scaffolds.” Acta Biomater. 18, 30 (2015). Several approaches to emulating ECM structural and mechanical cues in 3D engineered tissues have been used, including both aligned and unorganized nanofiber mats. Aligned and unorganized nanofiber mats feature high surface areas and fiber densities, but the high fiber density can make cell infiltration challenging, especially in thicker mats. Recent innovations have allowed for the collection of aligned electrospun fiber arrays, but the fabrication of more complex patterns remains a challenge.

Hwang et al., J. Biomed. Mater. Res. A, 104, 1017 (2016) describes poly(ε-caprolactone)/gelatin composite electrospun scaffolds with porous crater-like structures for tissue engineering. Several approaches to emulating ECM structural and mechanical cues in 3D engineered tissues have been used, including both aligned and unorganized nanofiber mats. Aligned and unorganized nanofiber mats feature high surface areas and fiber densities, but the high fiber density can make cell infiltration challenging, especially in thicker mats. Recent innovations have allowed for the collection of aligned electrospun fiber arrays, but the fabrication of more complex patterns remains a challenge. Aligned arrays of microfibers composed of collagen, fibrin, and other natural polymers have been used as scaffolds in a variety of tissue systems.

Xu et al., J. Mech. Behav. Biomed. Mater., 65, 428 (2017) describes the preparation and characterization of electrospun alginate/PLA nanofibers as tissue engineering material by emulsion eletrospinning. Several approaches to emulating ECM structural and mechanical cues in 3D engineered tissues have been used, including both aligned and unorganized nanofiber mats. Aligned and unorganized nanofiber mats feature high surface areas and fiber densities, but the high fiber density can make cell infiltration challenging, especially in thicker mats. Recent innovations have allowed for the collection of aligned electrospun fiber arrays, but the fabrication of more complex patterns remains a challenge.

Park et al., “Surface modification of biodegradable electrospun nanofiber scaffolds and their interaction with fibroblasts.” J. Biomater. Sci. Polym. Ed., 18, 369 (2007). Several approaches to emulating ECM structural and mechanical cues in 3D engineered tissues have been used, including both aligned and unorganized nanofiber mats. Aligned and unorganized nanofiber mats feature high surface areas and fiber densities, but the high fiber density can make cell infiltration challenging, especially in thicker mats. Recent innovations have allowed for the collection of aligned electrospun fiber arrays, but the fabrication of more complex patterns remains a challenge.

Wu et al., “Preparation of aligned porous gelatin scaffolds by unidirectional freeze-drying method.” Acta Biomater., 6, 1167 (2010). Several approaches to emulating ECM structural and mechanical cues in 3D engineered tissues have been used, including aligned pores through directional freezing. Directional freezing has been used to create uniform, anisotropic pore arrays in a range of common natural and synthetic scaffold materials. The aligned pores improve cell infiltration and impart mechanical and structural anisotropy. However, directional controlled rate freezing features a somewhat limited design space in terms of the morphologies that can be created.

Zhang & Cooper, “Aligned porous structures by directional freezing.” Adv. Mater., 19, 1529 (2007). Several approaches to emulating ECM structural and mechanical cues in 3D engineered tissues have been used, including aligned pores through directional freezing. Directional freezing has been used to create uniform, anisotropic pore arrays in a range of common natural and synthetic scaffold materials. The aligned pores improve cell infiltration and impart mechanical and structural anisotropy. However, directional controlled rate freezing features a somewhat limited design space in terms of the morphologies that can be created.

Deville, “Freeze-casting of porous biomaterials: structure, properties and opportunities.” Materials, 3, 1913 (2010). Several approaches to emulating ECM structural and mechanical cues in 3D engineered tissues have been used, including aligned pores through directional freezing. Directional freezing has been used to create uniform, anisotropic pore arrays in a range of common natural and synthetic scaffold materials. The aligned pores improve cell infiltration and impart mechanical and structural anisotropy. However, directional controlled rate freezing features a somewhat limited design space in terms of the morphologies that can be created.

Zhang et al., “Aligned two- and three-dimensional structures by directional freezing of polymers and nanoparticles.” Nat. Mater., 4, 787 (2005). Several approaches to emulating ECM structural and mechanical cues in 3D engineered tissues have been used, including aligned pores through directional freezing. 3D printed scaffolds offer high reproducibility and a broad design space, but compromises must be made in terms of either resolution or scaffold polymer, often resulting in excluding natural polymers. The aligned pores improve cell infiltration and impart mechanical and structural anisotropy.

Madden et al., “Proangiogenic scaffolds as functional templates for cardiac tissue engineering.” Proc. Natl. Acad. Sci. U.S.A, 107, 15211 (2010). Several approaches to emulating ECM structural and mechanical cues in 3D engineered tissues have been used, including sphere- and rod-templated scaffolds. 3D printed scaffolds offer high reproducibility and a broad design space, but compromises must be made in terms of either resolution or scaffold polymer, often resulting in excluding natural polymers.

Bhrany et al., “Evaluation of a sphere-templated polymeric scaffold as a subcutaneous implant.” JAMA Facial Plast. Surg., 15, 29 (2013). Several approaches to emulating ECM structural and mechanical cues in 3D engineered tissues have been used, including sphere- and rod-templated scaffolds. 3D printed scaffolds offer high reproducibility and a broad design space, but compromises must be made in terms of either resolution or scaffold polymer, often resulting in excluding natural polymers.

Chia & Wu, “Recent advances in 3D printing of biomaterials.” J. Biol. Eng., 9, 4 (2015). Several approaches to emulating ECM structural and mechanical cues in 3D engineered tissues have been used, including 3D printed scaffolds. 3D printed scaffolds offer high reproducibility and a broad design space, but compromises must be made in terms of either resolution or scaffold polymer, often resulting in excluding natural polymers.

Derakhshanfar et al., “3D bioprinting for biomedical devices and tissue engineering: a review of recent trends and advances.” Bioact. Mater., 3, 144 (2018). Several approaches to emulating ECM structural and mechanical cues in 3D engineered tissues have been used, including 3D printed scaffolds. 3D printed scaffolds offer high reproducibility and a broad design space, but compromises must be made in terms of either resolution or scaffold polymer, often resulting in excluding natural polymers.

Mosadegh et al., “Current progress in 3D printing for cardiovascular tissue engineering.” Biomed. Mater. 10, 034002 (2015). Several approaches to emulating ECM structural and mechanical cues in 3D engineered tissues have been used, including 3D printed scaffolds. 3D printed scaffolds offer high reproducibility and a broad design space, but compromises must be made in terms of either resolution or scaffold polymer, often resulting in excluding natural polymers.

Jung et al., “Solid organ fabrication: comparison of decellularization to 3D bioprinting.” Biomater. Res., 20, 27 (2016). Several approaches to emulating ECM structural and mechanical cues in 3D engineered tissues have been used, including 3D printed scaffolds. 3D printed scaffolds offer high reproducibility and a broad design space, but compromises must be made in terms of either resolution or scaffold polymer, often resulting in excluding natural polymers.

Khorshidi et al., J. Tissue Eng. Regen. Med., 10, 715 (2016). This publication is a review of key challenges of electrospun scaffolds for tissue-engineering applications. Aligned and unorganized nanofiber mats feature high surface areas and fiber densities, but the high fiber density can make cell infiltration challenging, especially in thicker mats.

Jiang et al., “Expanded 3D nanofiber scaffolds: cell penetration, neovascularization, and host response.” Adv. Healthc. Mater., 5, 2993 (2016). Aligned and unorganized nanofiber mats feature high surface areas and fiber densities, but the high fiber density can make cell infiltration challenging, especially in thicker mats.

Vaquette & Cooper-White, “Increasing electrospun scaffold pore size with tailored collectors for improved cell penetration.” Acta Biomater., 7, 2544 (2011). Aligned and unorganized nanofiber mats feature high surface areas and fiber densities, but the high fiber density can make cell infiltration challenging, especially in thicker mats.

Fan et al., “Optimal elastomeric scaffold leaflet shape for pulmonary heart valve leaflet replacement.” J. Biomech., 46, 662 (2013). Recent innovations have allowed for the collection of aligned electrospun fiber arrays

Masoumi et al., “Tri-layered elastomeric scaffolds for engineering heart valve leaflets.” Biomaterials, 35, 7774 (2014). Recent innovations allow for the collection of aligned electrospun fiber arrays.

Masoumi N et al., “Electrospun PGS:PCL microfibers align human valvular interstitial cells and provide tunable scaffold anisotropy.” Adv. Healthc. Mater., 3, 929 (2014). Recent innovations allow for the collection of aligned electrospun fiber arrays.

Bai et al., “Biomimetic gradient scaffold from ice-templating for self-seeding of cells with capillary effect.” Acta Biomater., 20, 113 (2015). The aligned pores improve cell infiltration and impart mechanical and structural anisotropy.

Kroehne et al., “Use of a novel collagen matrix with oriented pore structure for muscle cell differentiation in cell culture and in grafts.” J. Cell Mol. Med., 12, 1640 (2008). The aligned pores improve cell infiltration and impart mechanical and structural anisotropy.

Cornwell & Pins, “Discrete crosslinked fibrin microthread scaffolds for tissue regeneration.” J. Biomed. Mater. Res. A., 82A, 104 (2007). Aligned arrays of microfibers composed of collagen, fibrin, and other natural polymers have been used as scaffolds in a variety of tissue systems.

Yodmuang et al., “Silk microfiber-reinforced silk hydrogel composites for functional cartilage tissue repair.” Acta Biomater., 11, 27 (2015). Aligned arrays of microfibers composed of collagen, fibrin, and other natural polymers have been used as scaffolds in a variety of tissue systems.

Kumar et al., “Collagen-based substrates with tunable strength for soft tissue engineering.” Biomater Sci 1 (2013).

Kashiwabuchi et al., “Development of absorbable, antibiotic-eluting sutures for ophthalmic surgery.” Transl. Vis. Sci. Technol., 6, 1 (2017). Aligned arrays of microfibers composed of collagen, fibrin, and other natural polymers have been used as wound dressings and sutures.

Zhao et al., “Wound dressings composed of copper-doped borate bioactive glass microfibers stimulate angiogenesis and heal full-thickness skin defects in a rodent model.” Biomaterials, 53, 379 (2015). Aligned arrays of microfibers composed of collagen, fibrin, and other natural polymers have been used as wound dressings and sutures.

Chrobak et al., “Design of a fibrin microthread-based composite layer for use in a cardiac patch.” ACS Biomater. Sci. Eng., 3, 1394 (2017). Overlapping sets of natural polymer microfibers have also been used to create more sophisticated fibrous tissue scaffolds and therapeutic biomaterials with unique and tunable properties.

Caves et al., “The use of microfiber composites of elastin-like protein matrix reinforced with synthetic collagen in the design of vascular grafts.” Biomaterials 31, 7175 (2010). Overlapping sets of natural polymer microfibers have also been used to create more sophisticated fibrous tissue scaffolds and therapeutic biomaterials with unique and tunable properties.

Rajan et al., Nat. Protoc. 1, 2753 (2007) describes the preparation of ready-to-use, storable and reconstituted type I collagen from rat tail tendon for tissue engineering applications using a “twist-and-pull” method.

Caves et al., “Fibrillogenesis in continuously spun synthetic collagen fiber.” J. Biomed. Mater. Res. B., Appl. Biomater., 93, 24 (2010).

Kaiser et al., “Optimizing blended collagen-fibrin hydrogels for cardiac tissue engineering with human iPSC-derived cardiomyocytes.” ACS Biomater. Sci. Eng., 5, 887 (2019).

Baselt et al., “Subfibrillar structure of type I collagen observed by atomic force microscopy.” Biophys. J., 65, 2644 (1993).

Bozec et al., “Collagen fibrils: nanoscale ropes. Biophys J 92, 70 (2007

Zeugolis et al., “Electro-spinning of pure collagen nano-fibres—just an expensive way to make gelatin?” Biomaterials, 29, 2293 (2008).

Raub et al., “Predicting bulk mechanical properties of cellularized collagen gels using multiphoton microscopy.” Acta Biomater., 6, 4657 (2010).

Cook & McDonagh, “Measurement of muscle and tendon stiffness in man.” Eur. J. Appl. Physiol., 72, 380 (1996).

Iivarinen et al., “Experimental and numerical analysis of soft tissue stiffness measurement using manual indentation device—significance of indentation geometry and soft tissue thickness.” Skin Res. Technol., 20, 347 (2014).

Iivarinen et al., “Experimental and computational analysis of soft tissue stiffness in forearm using a manual indentation device.” Med. Eng. Phys., 33, 1245 (2011).

Capulli et al., “Fibrous scaffolds for building hearts and heart parts.” Adv. Drug Deliv. Rev., 96, 83 (2016).

Korinek et al., “Two-dimensional strain—a Doppler-independent ultrasound method for quantitation of regional deformation: validation in vitro and in vivo.” J. Am. Soc. Echocardiogr., 18, 1247 (2005).

Maganaris & Paul, “In vivo human tendon mechanical properties.” J. Physiol., 521, 307 (1999).

Cheng et al., “An electrochemical fabrication process for the assembly of anisotropically oriented collagen bundles.” Biomaterials, 29, 3278 (2008).

Pins et al., “Self-assembly of collagen fibers. Influence of fibrillar alignment and decorin on mechanical properties.” Biophys. J., 73, 2164 (1997).

Park et al., “The significance of pore microarchitecture in a multi-layered elastomeric scaffold for contractile cardiac muscle constructs.” Biomaterials, 32, 1856 (2011).

Neal et al., “Three-dimensional elastomeric scaffolds designed with cardiac-mimetic structural and mechanical features.” Tissue Eng. Part A., 19, 793 (2013).

Vollert et al., “In vitro perfusion of engineered heart tissue through endothelialized channels.” Tissue Eng. Part A., 20, 854 (2013).

Lovett et al., “Vascularization strategies for tissue engineering.” Tissue Eng. Part B Rev., 15, 353 (2009).

Sarig et al., “Pushing the envelope in tissue engineering: ex vivo production of thick vascularized cardiac extracellular matrix constructs.” Tissue Eng. Part A., 21, 1507 (2015).

Dippold et al., “Novel approach towards aligned PCL-Collagen nanofibrous constructs from a benign solvent system.” Mater. Sci. Eng. C., 72, 278 (2017).

Chakrapani et al., “Electrospinning of type I collagen and PCL nanofibers using acetic acid.” J. Appl. Polym. Sci., 125, 3221 (2012).

Haaparanta et al., “Preparation and characterization of collagen/PLA, chitosan/PLA, and collagen/chitosan/PLA hybrid scaffolds for cartilage tissue engineering.” J. Mater. Sci. Mater. Med., 25, 1129 (2014).

Sadeghi-Avalshahr et al., Regen Biomater 4, 309 (2017) describes the synthesis and characterization of collagen/PLGA biodegradable skin scaffold fibers.

De Vrieze et al., J. Mater. Sci., 44, 1357 (2008) describes electrospinning methods.

Pelipenko J et al., Int. J. Pharm. 456, 125 (2013) describes electrospinning methods.

Kaiser et al., “Digital Design and Automated Fabrication of Bespoke Collagen Microfiber Scaffolds.” Tissue Eng. Part C Methods, 25(11), 687-700. (Nov. 1, 2019).

### Machine

The subject innovation is now described with reference to the drawings, wherein like reference numerals are used to refer to like elements throughout. In the following description, for purposes of explanation, numerous specific details are set forth to provide a thorough understanding of the invention. It may be evident, however, that the invention may be practiced without these specific details. In other instances, well-known structures and devices are shown in block diagram form to facilitate describing the invention.

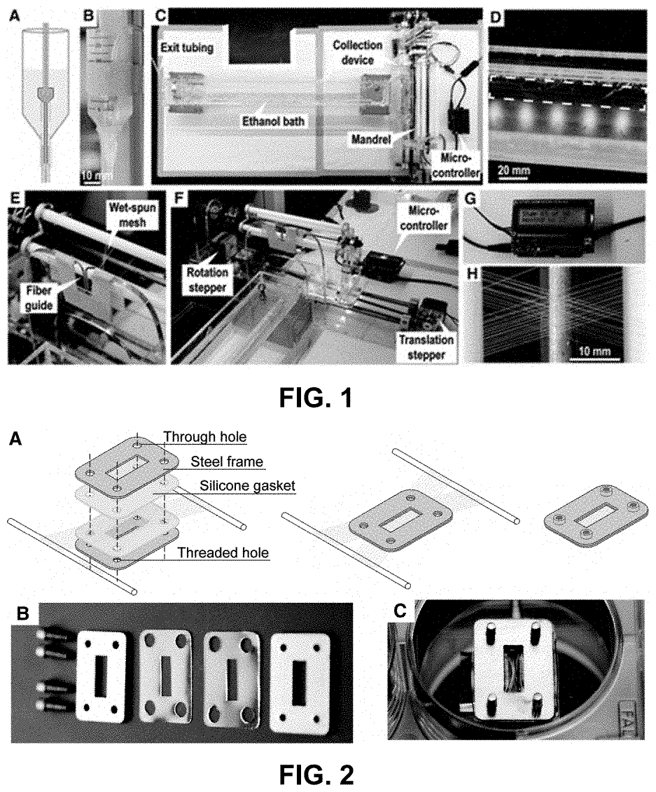

Referring to FIG. 1(A), a collagen fiber wet spinning and mesh device is shown. Across-sectional schematic of the spinneret composed of a 22-gauge syringe needle (red) is inserted into the needle cap. Collagen enters the spinneret through syringe pump extrusion and forms a co-axial flow system with wet-spin buffer descending from the buffer reservoir. See, FIG. 1(B).

Referring to FIG. 1(C), this overhead view of the bath and collector shows the elements of the device. The continuous collagen fiber (on the left side of the device) enters into the bath filled with 70% ethanol, which washes away residual polyethylene glycol and facilitates fiber drying. The fiber is pulled through the length of the bath, threaded through a fiber guide, and laid onto the mandrel of the collection device. FIG. 1(D) provides a close-up view of a continuous collagen fiber traveling through the 70% ethanol bath. The dotted line box indicates the location of the continuous collagen microfiber. FIG. 1(E) provides a close-up view of the fiber guide and the collection mandrel. The fiber collector consists of a fiber guide and the mandrel on a translating platform. See, FIG. 1(F). Two stepper motors, driven by an Arduino microcontroller, direct the rotation and translation of the mandrel. An Arduino microcontroller (see FIG. 1(G)) controls the collection device and executes mesh protocols. FIG. 1(H) provides a 30° collagen microfiber mesh on the mandrel. After the mesh is formed on the collection mandrel, it must be captured in a frame to maintain the precise geometry to be removed from the mandrel and used in downstream applications.

Collagen mesh capture frames are shown in FIG. 2. FIG. 2(A) shows that silicone gaskets and steel support frames are placed on opposite sides of a collagen microfiber mesh on the collection mandrel, the frames are collapsed together, and screws are put in place to hold the frame together. FIG. 2(B) shows screws, frames, and gaskets used in the capture process. FIG. 2(C) shows a captured mesh in the well of a six-well plate and ready for hydrogel/cell casting.

Several elements for the assembly of the device (the wet spinning bath) shown in FIG. 1C is shown in FIG. 6, which is a set of schematics showing laser cut ¼-inch acrylic parts for fiber collector and mandrel. These schematics are for reference only. Persons of ordinary skill in the art can use provided .ai files when laser cutting.

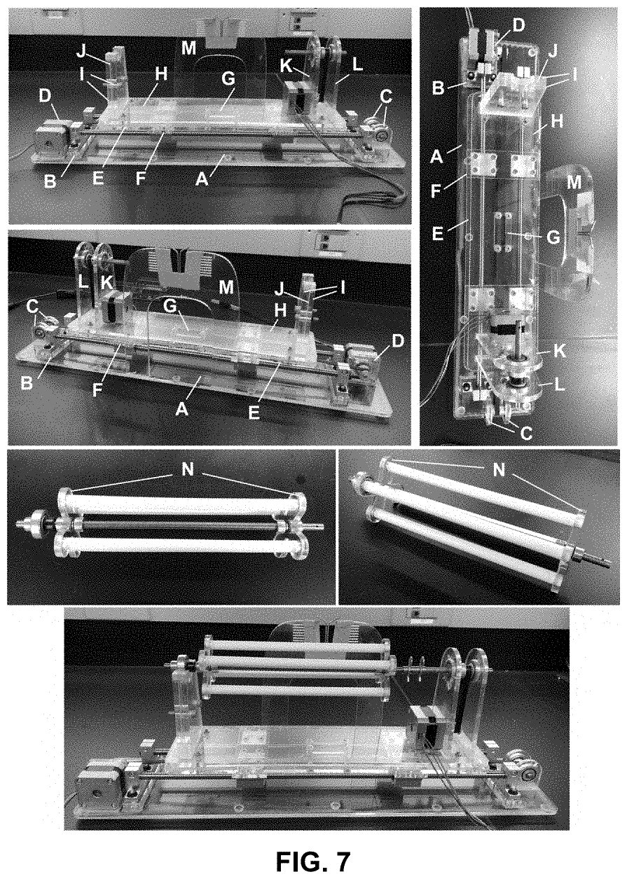

FIG. 7 is a set of photographs showing reference images for the constructed fiber collector and mandrel. The letters in FIG. 7 refer to the corresponding elements in FIG. 6.

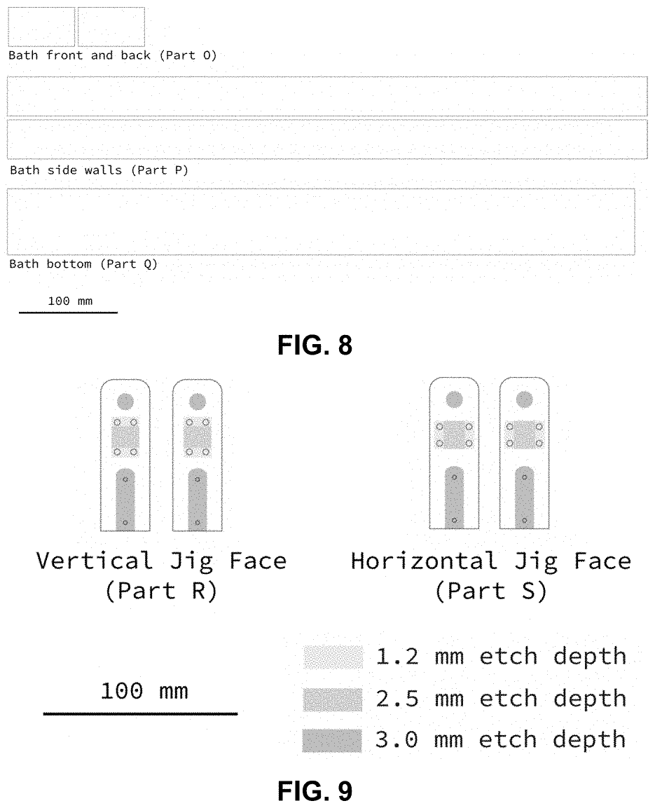

FIG. 8 is a set of schematics showing laser cut acrylic parts for the device (the wet spinning bath) shown in FIG. 1C. These schematics are for reference only. Persons of ordinary skill in the art can use provided .ai files when laser ting for making embodiments of different sizes. Note that the metal fixtures described in this specification are unnecessary for the methods described in this manuscript.

FIG. 9 is a set of schematics showing laser cut acrylic parts for an alignment jig that helps align and hold the capture frames in place to preserve the collagen microfiber mesh design. These schematics are for reference only. Persons of ordinary skill in the art can use provided .ai files when laser cutting for making embodiments of different sizes.

FIG. 10 is a pair of photographs showing a reference image for an assembled alignment jig.

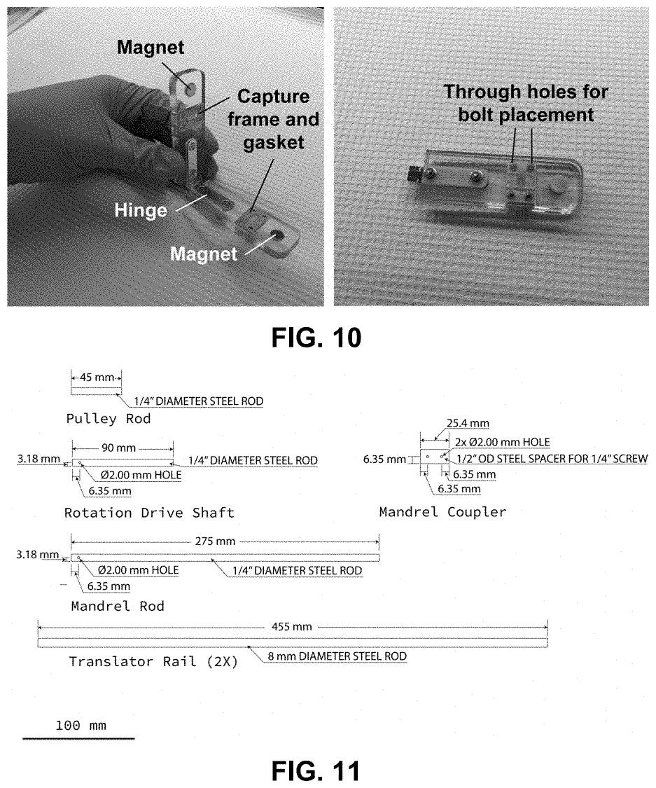

FIG. 11 is a set of schematics showing design plans for machined steel fiber collector parts.

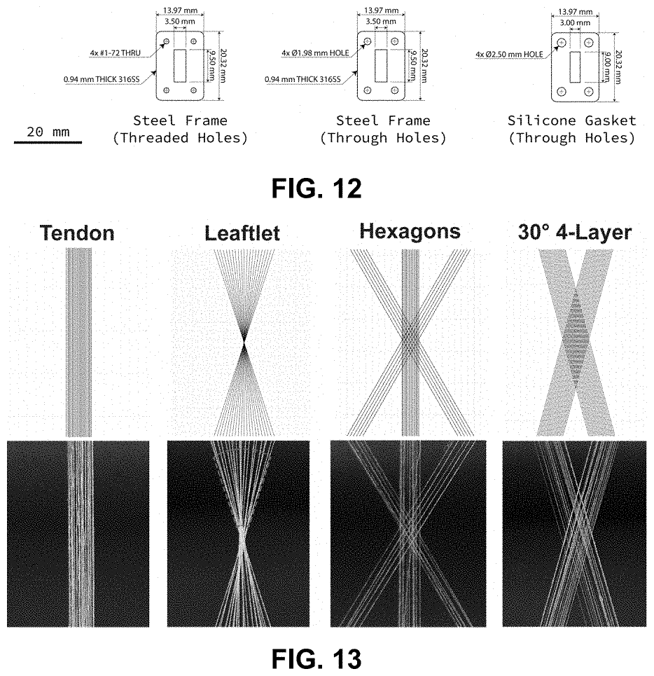

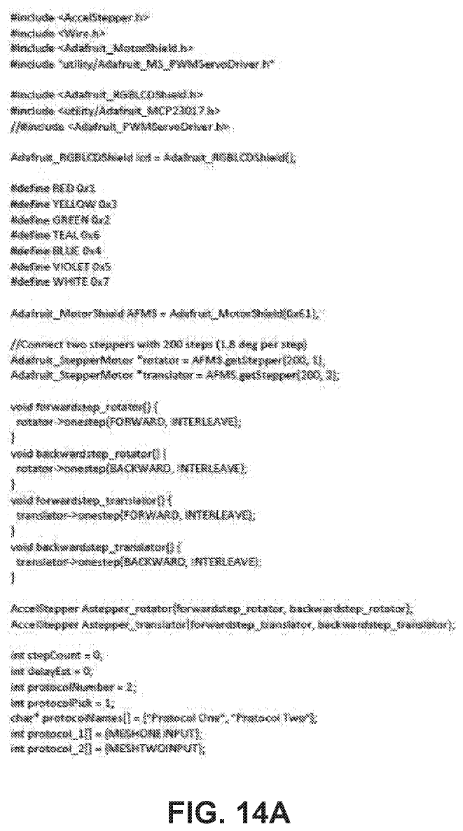

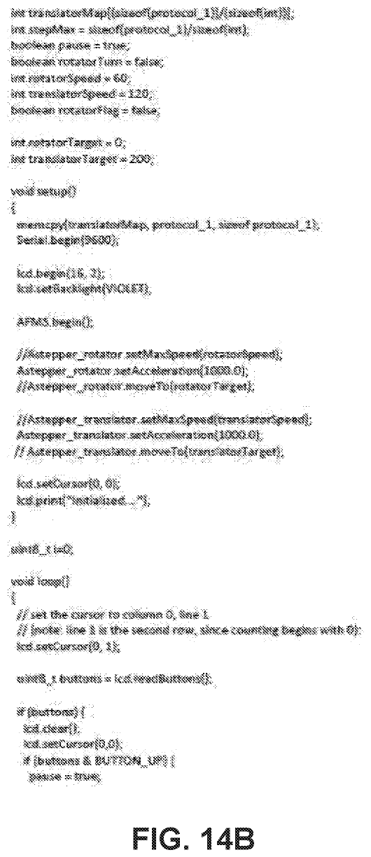

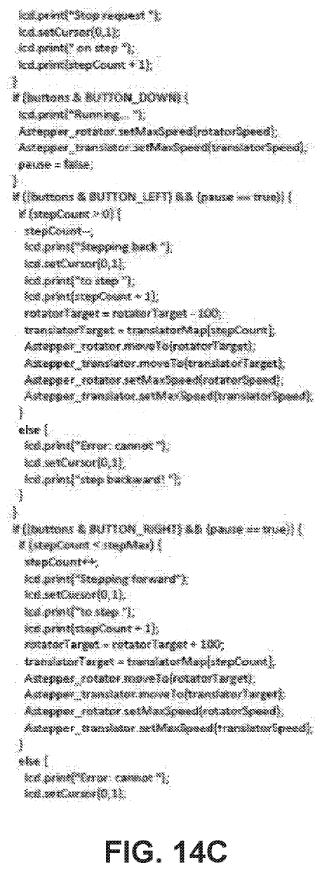

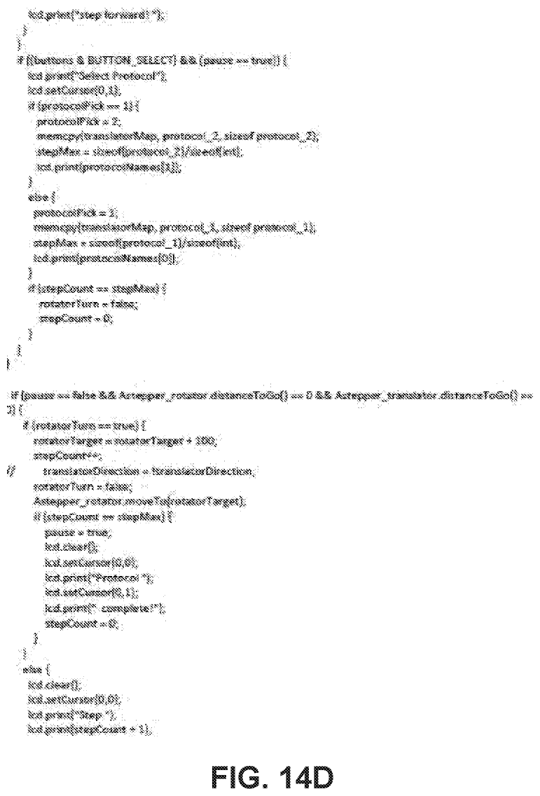

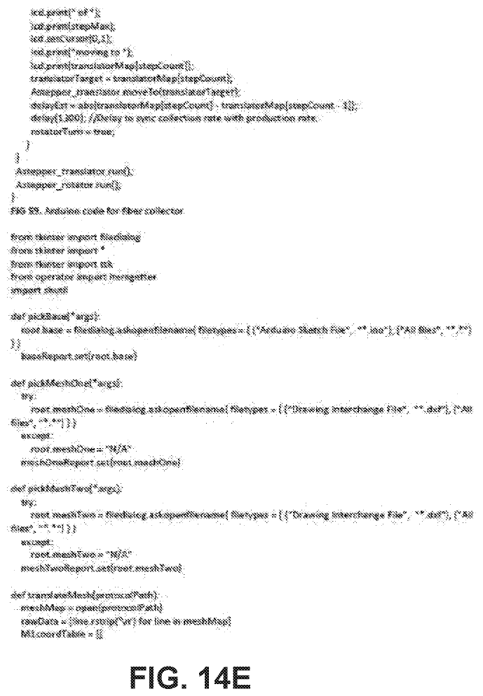

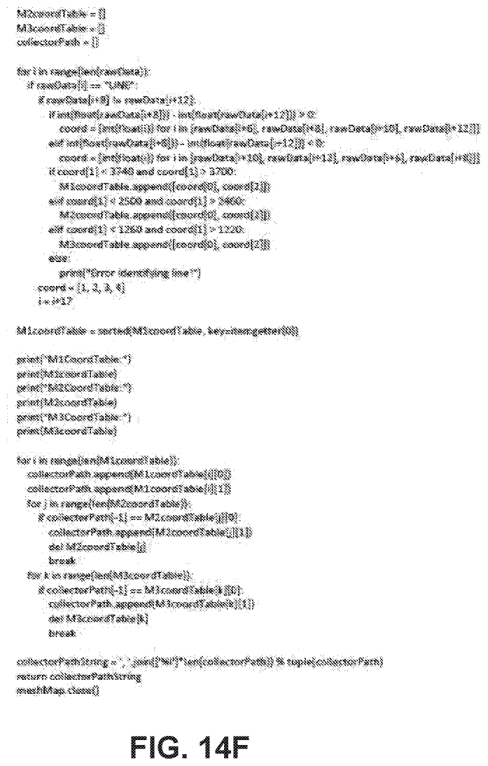

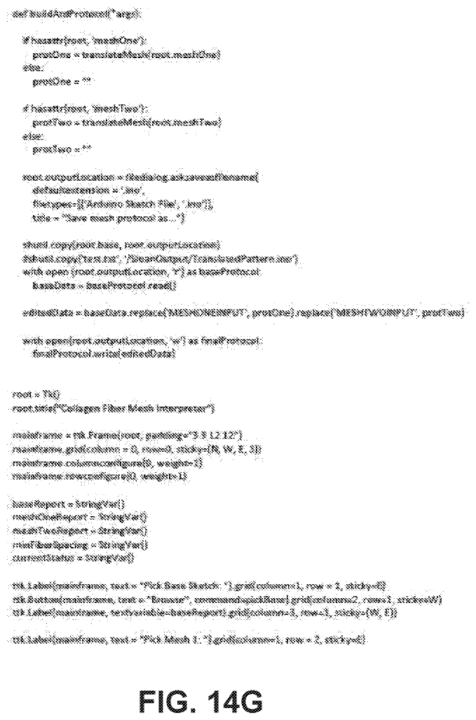

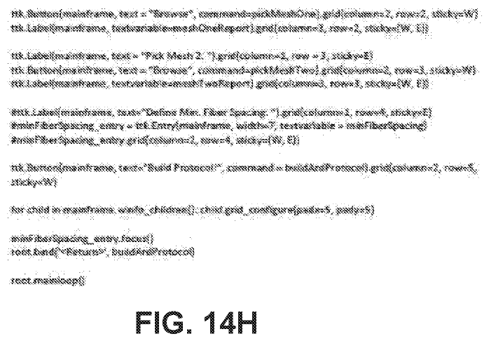

FIG. 12 is a set of schematics showing design plans for steel frames and silicone gaskets.

All acrylic to acrylic connections between parts are made by sanding with 150 grit sandpaper and then gluing with cyanoacrylate adhesive. See FIGS. 6-12 for laser cutting designs and reference during assembly. Bolts and screws are referred to here by their thread size.

TABLE 1 lists further part details including exemplary parts for fiber collector, wet spinning bath, and alignment jig.

TABLE 1
List of required parts for fiber collector and
wet spinning bath, alignment jig, and collection frames.

| Part | Vendor | Part Number | Quantity |
|---|---|---|---|
| Fiber Collector and Mandrel | | | |
| Linear Rail Shaft Guide/Support—8 mm | Adafruit | 1182 | 4 |
| diameter—SK8 | | | |
| Linear Bearing Platform (Small)—8 mm | Adafruit | 1179 | 4 |
| Diameter—SC8UU | | | |
| Stepper motor—NEMA—17 size—200 | Adafruit | 324 | 2 |
| steps/rev, 12 V 350 mA | | | |
| Adafruit Motor/Stepper/Servo Shield for | Adafruit | 1438 | 1 |
| Arduino v2 Kit—v2.3 | | | |
| RGB LCD Shield Kit w/16 × 2 Character | Adafruit | 716 | 1 |
| Display | | | |
| Arduino UNO R3 | Arduino | A000066 | 1 |
| In-line power switch for 2.1 mm barrel jack | Adafruit | 1125 | 1 |
| 9V DC 1000 mA regulated switching power | Adafruit | 63 | 1 |
| adapter | | | |
| Waterproof Polarized 4-Wire Cable Set | Adafruit | 744 | 2 |
| Shield stacking headers for Arduino (R3 | Adafruit | 85 | 1 |
| Compatible) | | | |
| Aluminum GT2 Timing Pulley—6 mm | Adafruit | 1251 | 1 |
| Belt—20 Tooth 5 mm Bore | | | |
| Aluminum GT2 Timing Pulley—6 mm | Adafruit | 1252 | 1 |
| Belt—20 Tooth 8 mm Bore | | | |
| Timing Belt GT2 Profile—2 mm pitch— | Adafruit | 1184 | 1 |
| 6 mm wide 1164 mm long | | | |
| Steel Ball Bearing Plain Open for ¼″ Shaft | McMaster- | 6383K22 | 6 |
| Diameter, 1″ OD, 5/16″ Width | Carr | | |
| Set Screw Shaft Collar for ¼″ Diameter, | McMaster- | 9414T6 | 10 |
| Black-Oxide Steel | Carr | | |
| Linear Motion Shaft, 1055 Carbon Steel, | McMaster- | 6112K46 | 2 |
| 8 mm Diameter, 600 mm Long | Carr | | |
| Mcl and XL Series Timing-Belt Pulley ¼″ | McMaster- | 1375K39 | 2 |
| Belt Width, 0.685 OD, 20 Teeth | Carr | | |
| Multipurpose 4140/4142 Alloy Steel, Rod | McMaster- | 8927K18 | 2 |
| ¼″, 2 ft | Carr | | |
| Trapezoidal Tooth Urethane Timing Belt, | McMaster- | 1679K86 | 1 |
| .080″ Pitch, Trade Size 90 mxl, 7.2″ Outer | Carr | | |
| Circle, ¼″ Wide | | | |
| Tube Made of Teflon ® PTFE, 3/8″ OD × | McMaster- | 8547K11 | 4 |
| ¼″ ID 2 ft | Carr | | |
| Polyurethane Rubber Adhesive-Back | McMaster- | 95495K673 | 1 |
| Bumper, Domed, 7/16″ OD, 13/64″ High, | Carr | | |
| Durometer 50 A, Clear | | | |
| Oil-Resistant Buna-N O-Ring, 1/16 | McMaster- | 9452K16 | 1 |
| Fractional Width, Dash Number 008 | Carr | | |
| 18-8 Stainless Steel Unthreaded Spacer, ½″ | McMaster- | 92320A669 | 1 |
| OD, 1″ Long, for ¼″ Screw Size | Carr | | |
| 18-8 Stainless Steel Hairpin Cotter Pin, for | McMaster- | 92391A135 | 1 |
| ⅜″ to ½″ Clevis Diameter, 5/64″ Wire | Carr | | |
| Diameter | | | |
| Class 12.9 Socket Head Cap Screw, Zinc- | McMaster- | 95263A402 | 10 |
| Coated Alloy Steel, M5 Thread, 25 mm | Carr | | |
| Long, .8 mm Pitch | | | |
| Uncoated Class 8 Steel Serrated-Flange | McMaster- | 94920A300 | 10 |
| Locknut M5 × 0.8 Thread, 11.2 mm Flange | Carr | | |
| Diameter, 4.3 mm Overall Height | | | |
| Coated Alloy Steel Socket Head Cap Screw | McMaster- | 91274A105 | 8 |
| M3 Thread, 10 mm Long | Carr | | |
| 18-8 Stainless Steel Low-Profile Socket Head | McMaster- | 92855A416 | 24 |
| Screws M4 × 0.7 mm Thread, 16 mm Long | Carr | | |
| Type 18-8 Stainless Steel Thin Hex Nut-DIN | McMaster- | 90710A035 | 8 |
| 439B M4 × 0.7 Thread Size, 7 mm Wide, | Carr | | |
| 2.2 mm High | | | |
| Alignment Jiq | | | |
| HIGHPOINT Side Rail Hinge Solid Brass | Woodcraft | 161694W | 2 |
| 316 Stainless Steel Hex Drive Flat Head | McMaster- | 90585A120 | 8 |
| Screw 82 Degree Countersink Angle, 2-56 | Carr | | |
| Thread Size, ⅜″ Long | | | |
| 18-8 Stainless Steel Hex Nut 2-56 Thread | McMaster- | 91841A003 | 8 |
| Size | Carr | | |
| 316 Stainless Steel Washer for Number 2 | McMaster- | 90107A003 | 8 |
| Screw Size, 0.094″ ID, 0.25″ OD | Carr | | |
| High-Pull Rare Earth Magnetic Disc | McMaster- | 5862K52 | 4 |
| Neodymium 0.1″ Thick, ¼″ Diameter | Carr | | |
| Collection Frames | | | |
| 18-8 Stainless Steel Socket Head Screw 1-72 | McMaster- | 92196A066 | 4/frame |
| Thread Size, ¼″ Long | Carr | | set |

Laser etch and cut all parts in the included laser design files out of ¼″ thick acrylic. See FIG. 6, FIG. 8, and FIG. 9. Target etching depths are described in each document. Specific power, speed, and dots per inch (DPI) settings are laser dependent. A Universal Laser System PLS 6.75 equipped with a 75 Watt laser cuts through ¼″ acrylic cleanly with power, speed, and DPI settings of 100%, 1, and 1000, etches to a depth of 2.00 mm with settings of 100%, 10, and 1000, and etches to a depth of 3.00 mm with settings of 100%, 7, and 1000.

Persons of ordinary skill in the tissue engineering art then prepare the required machined components. See, FIG. 11 and FIG. 12 for details. Using a local machine shop to match required specifications is recommended. Steel capture frames are best cut by an industrial laser cutter followed by tapping the 1-72 threaded holes.

Persons of ordinary skill in the tissue engineering art assemble the fiber collector translator base by sanding the bottom edge of the translator stepper motor mount (Part D) and gluing into place on the translator base (Part A). Similarly, sand and glue two stacked rail shaft risers (Part B) into the matching etched slots in the translator base, with M5 bolts in place to ensure screw holes are aligned. Press fit and glue the two ball bearings into the openings in the pulley mounts (Part C), sand the bottom of the mounts, and glue into place with the pulley rod inserted to ensure alignment. Install the NEMA-17 stepper motor with four M3 bolts through the motor mount. Install the four linear rail shaft guides with M5 bolts. Attach the 5 mm GT2 timing pulley to the shaft of the translation stepper and the 8 mm GT2 timing pulley to the pulley shaft. Insert each 8 mm rail through each of the linear bearing platforms and one pair of rail guides. Tighten the rail guide screws to fix the rods in place. Attach the linear bearing mounts (Part F) and the translator platform (Part E) to the linear bearing platforms with 16 M4 screws. Prepare the timing belt grip (Part G) by cutting a 5 cm length of the GT2 timing belt and gluing into the etched trough, so the teeth of the belt are exposed. Finally, install the translator timing belt by wrapping the remainder of the GT2 timing belt around the two translator timing pulleys, fixing the loose ends in place between the bottom of the translator platform (Part E), and the timing belt grip with four M4 bolts, cutting away excess.

Assemble the fiber collector rotator by stacking the inner mandrel support (Part J) between the two outer mandrel supports (Part I) so the etched regions face each other. Fix in place with two M5 bolts before sanding the bottom and gluing into the matching slot on the rotator base (Part H). Press fit and glue the two ball bearings into the matching openings on the rotator stepper mount (Part K) and rotator shaft support (Part L). Install one Mcl and XL series timing pulley on the rotator stepper motor and attach to the rotator stepper mount with four M3 screws. After the glue is dry, similarly sand the bottom and press fit these components into their matching slots on the rotator with the rotation drive shaft in place to ensure alignment. Install the other Mcl and XL series timing pulley on the rotation drive shaft and connect the two pulleys with the urethane timing belt.

Prepare one or more collection mandrels by press fitting the 37.5 cm long, ⅜″ diameter polytetrafluoroethylene (PTFE) rods into the four large openings of each of the mandrel caps (Part N). Teflon rods can be cut to size with a razor blade. Slide the mandrel caps, number 008 O-rings, shaft collars, and the end ball bearing onto the mandrel rod. Tighten the set screws of the shaft collars to fix the mandrel caps and ball bearing in place, as shown in FIG. 7, using the O-rings and shaft collars to hold the components in place.

Sand all four edges of the ethanol bath bottom (Part Q) and connection regions of the sides (Part P) and front and back (Part 0). After sanding, clamp the bath together and glue by distributing cyanoacrylate adhesive along all connections. After dry, seal all internal connections with silicone sealant to make the bath liquid tight.

Prepare the fiber guide support (Part M) by drilling two small holes 1 mm apart into the bottom of the top opening, sized to fit metal rods bent to form a narrow channel. See, FIG. 7. Catheter IV needles (14-gauge, 2.1 mm OD) work well.

Finally, complete the fiber collector by attaching the fiber collector rotator to the translator with four M4 bolts through the precut holes. Attach the fiber guide by sanding the bottom and gluing into the matching etched slots. Mount a collection mandrel with the mandrel coupler and cotter pins. Position the bath at the end of the fiber collector. Decreasing the distance between the bath and the collection mandrel may reduce fiber breaks during mesh collection. See, FIG. 1. Adhesive feet can optionally be attached to the bottom of the fiber collector and ethanol bath.

### Manufacture/Mesh Designs

Mesh fidelity depends on fiber spacing. See, EXAMPLE 4 and FIG. 4(A)-4(C) for information about fiber spacing. With 400 μm spacing, effective dry fiber diameters were consistent (51.26±11.16 μm, mean±SD). This variability also increased when spacing became small because of electrostatic interactions that drew adjacent fibers together (81.85±80.38 for 100 μm, mean±SD). Similar collagen wet spinning methods have been used to produce collagen fibers with dry diameters ranging from 20 to 150 μm. See, Caves et al., J. Biomed. Mater. Res. B., Appl. Biomater., 93, 24 (2010). However, the fiber diameter in this specification is associated with the wet spinning conditions that were found to minimize fiber breaks during mesh fabrication.

See EXAMPLE 3, EXAMPLE 4, and FIG. 5D and FIG. 5(E) for information about fiber angle.

Fibrous collagen composite scaffolds are viable tissue scaffolds as shown by the culture of hiPSC-derived cardiomyocytes on meshes prepared in both parallel and 30° fiber configurations. See, FIG. 5. Resident cells rapidly remodeled the bulk hydrogel and exerted stress on the collagen microfibers as shown by their deformation. See, FIGS. 6A and 6B. Fluorescent live/dead staining of cellularized constructs showed a high density of viable cardiomyocytes and few dead cells throughout the tissue. See, FIG. 5C. Immunohistochemistry confirmed that the resident cardiomyocytes maintained a striated morphology. See, FIG. 5D.

FIG. 13 shows mesh patterns designed via a digital graphical user interface (GUI) and translated into protocols executed by a custom mesh collection and organization device, examples of which are provided herein.

The device in this specification can produce meshes with fiber spacing about 100 μm, though in practice this is somewhat compromised by electrostatic interactions between adjacent fibers. Without more sophisticated equipment to mitigate these effects, relatively consistent fiber spacing can be achieved at 400 μm and higher fiber spacing. Meshes targeting smaller fiber spacing will simply have more variability in terms of this parameter, and the tolerance of this variability will depend greatly on the intended application. Conversely fiber angle, a parameter unaffected by electrostatic interactions, was shown to be highly consistent both within a single production run and across multiple production batches.

The simple method of mesh design and automated fabrication in this specification extends the advantages of automated biomaterial fabrication to a wide variety of uses.

### Methods of Manufacture

Isolation of Type I Collagen from Rat Tail Tendon.

Rat tail tendons are a relatively high purity source of type I collagen. Other tissues can be a source of type I collagen, such as bovine dermis, human cadaver tendon, and other vertebrate tissues.

This method of isolating type I collagen from rat tail tendon uses acid to extract full length telopeptide collagen. Rat tails are collected from Sprague-Dawley rats within thirty minutes of sacrifice. The tails are immediately frozen at −20° C. for up to six months. Then, the tails are thawed for two hours at room temperature and thoroughly washed with ethanol before tendon harvest.

The rat tendons are harvested using a “twist-and-pull” method described by Rajan et al., Nat. Protoc. 1, 2753 (2007). Briefly, a person of ordinary skill in the tissue engineering art can grasp rat tails both at the cut end of the tail and at a second point approximately 2.5 cm further down the tail (towards the tip of the tail) using a pair of clean, sturdy hemostats or needle-nose pliers in each hand. While the pliers closer to the tip of the tail are held firmly in place, the second pair of pliers is rotated around the axis of the tail while grasping the 2.5 cm section, causing the tissue and cartilage to break away. After 2-3 rotations, the 2.5 cm section can be pulled away and discarded, revealing the tail tendons. Exposed sections of tail tendons are cut with scissors into a beaker filled with about 250 mL of sterile phosphate-buffered saline (PBS) to wash off tissue debris and keep tendons hydrated. Isolation of the collagen-containing material by dissection and cell removal should be pursued for all sources of fibrillar collagen.

Washed tail tendons from approximately twelve tails are transferred to a 2 L beaker filled with 1.8 L of 0.1M acetic acid in deionized (DI) water for collagen extraction. The beaker is stirred at 120 rpm at 4° C. for seventy-two hours to solubilize the collagen.

Tissue debris is separated from the acid extracted collagen solution via centrifugation at 8,000×g at 4° C. for two hours. The supernatant containing the collagen is decanted into a beaker and the tissue debris pellets are discarded.

Collagen is precipitated from the acetic acid solution by the gradual addition of NaCl in deionized water while on a stir plate set to 200 rpm, until the collagen solution reaches a NaCl concentration of 4%. Stirring continues for one hour at 4° C., after which precipitated collagen is visible.

Precipitated collagen is isolated via centrifugation at 8,000×g at 4° C. for two hours. The supernatant is discarded. The pellets of collagen are then re-dissolved in 0.1M acetic acid on a stir plate set to 120 rpm at 4° C. over 72 hours. Using a small volume of acetic acid (about 75 mL for twelve rat tails) allows for preparing a high concentration stock solution. An additional 0.1M acetic acid can be added to dissolve the collagen.

After the dissolution of the collagen pellets is complete, a five mL aliquot of the collagen solution is lyophilized and weighed to determine the collagen concentration of the prepared solution (mass/volume). An additional 0.1M acetic acid is added to the remaining solubilized collagen to achieve a stock concentration of thirteen mg/mL. One can store collagen at 4° C. for up to six months.

Chloroform vapor can sterilize the collagen solution. A person of ordinary skill in the tissue engineering art can aliquot a volume of chloroform equal to 10% of the volume of collagen to be sterilized into the bottom of a conical tube or beaker. Carefully float the volume of collagen on top of the chloroform and store overnight at 4° C. The next day, collect the collagen solution, being careful not to take up any chloroform. Store at 4° C. for up to six months.

Designing Microfiber Mesh Protocols.

Mesh patterns are designed using an open source flowchart software program called “Dia” (https://sourceforge.net/projects/dia-installer/), which allows for simple “click and drag” placement of lines in a graphical user interface. A prepared Dia (.dia) data file with mesh windows matching the dimensions of those on the collection mandrels facilitates mesh design in each of the four mesh windows (3.5×19 cm). See, FIG. 7.

Mesh protocols designed in Dia are saved and exported as .dxf files (document exchange format). This format identifies the lines that compose the mesh design based on the x,y coordinates of their start and end points.

Mesh protocol .dxf files are translated into .ino Arduino format protocols using a Python script available for download. See, FIG. 13. Multiple mesh designs can be translated into a single .ino file which can be individually chosen during mesh collection.

Translated .ino format protocols are uploaded to the Arduino microcontroller with Motor Shield and RGB LCD Shield (Adafruit Industries, New York, N.Y.) via USB for mesh fabrication. The Arduino microcontroller and Motor Shield provide a simple means of manipulating the two stepper motors, while the RGB LCD Shield provides a user interface during mesh collection.

Wet Spinning Collagen Meshes.

Collagen is wet spun into microfibers using methods adapted from Caves et al., J. Biomed. Mater. Res. B., Appl. Biomater., 93, 24 (2010). Before the start of wet spinning, the outer surfaces of the four polytetrafluoroethylene (PTFE) rods on the collection mandrel are coated with Neoweld Contact Cement (Springfield Leather Company, Springfield, Mo., USA) and allowed to dry for 20 minutes. This adhesive remains tacky for days after drying, holding fibers in place as they are collected and maintaining mesh design fidelity.

Dissolved gases in the collagen solution may form bubbles that interrupt the continuous wet spun fiber. If this problem arises, the collagen solution can be degassed in a vacuum chamber for thirty minutes before wet spinning.

The 64×7 cm bath (FIG. 1C) is filled with 650 mL of 70% ethanol, so the depth in the bath is approximately 1.5 cm, allowing the fiber and tubing to remain submerged during collection.

The buffer reservoir of the spinneret (FIG. 1A, 1B) is filled with high viscosity wet spinning buffer solution (HV-WSB, 34.5 mM potassium phosphate monobasic, 85.2 mM sodium phosphate dibasic, 135 mM sodium chloride, 29.9 mM HEPES buffer, 8.57 mM polyethylene glycol (PEG) MW 35,000). Buffer flows into the one-meter length of 1.6 mm inner diameter PVC tubing is initiated by applying suction to the end of the tubing with a micropipette.

After HV-WSB exits the length of PVC tubing, thirteen mg/mL collagen I in 0.1 M acetic acid is extruded via syringe pump at 50 μL/min through a 0.4 mm inner diameter spinneret prepared from a 22-gauge syringe needle into the same length of 1.6 mm inner diameter PVC tubing, forming a coaxial flow system (FIG. 1B). Collagen precipitates from the thirteen mg/mL solution as it travels the length of the tubing.

The continuous collagen fiber exits into the 70% ethanol bath, which washes away residual buffer solution and aids in drying the fiber.

The collagen fiber is picked up with forceps, dragged through the length of the bath, and a length of about 20 cm is held in the air for about thirty seconds, or until the fiber is dry. The dry fiber is then threaded through the fiber guide (FIG. 1F) and onto the collection mandrel rods.

The collection protocol is initiated on the Arduino microcontroller. See, FIG. 1G. When the mandrel rotates, the fiber is pulled through the fiber guide and onto the mandrel in the prescribed pattern. Drying time between mandrel rotations is an important parameter that can be modified in the digital collection protocol.

After the mesh protocol is complete, the mandrel is removed from the collection device for mesh capture.

Capturing and Embedding Wet Spun Collagen Meshes.

Mesh capture frames are stainless steel rectangular frames with a window matching the desired size of the composite tissue construct (, 3×9 mm). Paired with gaskets of similar dimensions, the frames hold the fibers in place during capture, embedding/casting, and cell culture. See, FIG. 2A.

Before mesh capture, 316 stainless steel frames, matching PDMS gaskets, 316 stainless steel screws (McMaster-Carr), and a matching hex key are autoclaved. Half of the stainless-steel frames feature 1-72 threaded holes, while the other half feature larger 1.98 mm diameter through holes, allowing the frames to be screwed together with ¼″ long 1-72 threaded screws.

The frame alignment jig is thoroughly sprayed with ethanol and is placed inside a biosafety cabinet along with the autoclaved tools. The frame alignment jig is composed of an upper half and a lower half (prepared from laser cut ¼″ thick acrylic plastic) connected by a metal hinge (HIGHPOINT Model 161694W). PDMS plugs sized to fit the windows of the capture frames hold the frames in place during capture. The upper half of the jig features four through holes so screws can be put in place while the frame is closed.

Aseptically, the stainless-steel capture frames are loaded onto the press fit PDMS plugs of the alignment jig with a frame with threaded holes on the lower half of the jig, and frame with through holes on the upper half of the jig.

The alignment jig is carefully positioned on either side of a mesh on the collection mandrel, with the region of interest aligned with the window of the lower capture frame. After positioned appropriately, the jig is closed and held in place helped by magnets.

Screws are inserted through the holes in the alignment jig and are screwed into place in the frame helped by the hex key. After all four screws are place, the alignment jig can be opened, and the capture mesh can be removed. Store captured meshes in a sterile vessel for up to two weeks before use. See, FIG. 2C.

Immediately before casting, while wearing sterile gloves, PDMS plugs are inserted into one window of each frame set. The plugs should be sized to press-fit into the stainless-steel windows. The frames are placed in an untreated six-well plate so the plugs contact the bottom of the plate and keep the frames and bolts suspended.

A collagen hydrogel casting mix is pipetted through the open window in the stainless-steel frame, using the tip of the pipette to guide the solution into the corners and edges of the window. With acellular constructs, 2.0 mg/mL collagen I in phosphate-buffered saline (pH 7.4) was used because of the poor handling properties of lower concentrations of collagen when uncompacted by resident cells. For cellularized constructs, 1.2 mg/mL collagen I in cell media is most effective, as shown by Kaiser et al., ACS Biomater. Sci. Eng., 5, 887 (2019).

After casting is complete, the 6-well plate is placed in a 37° C. incubator for thirty minutes or until the collagen hydrogel has become opaque. Enough phosphate-buffered saline or cell media for cellularized constructs is then added to each well to ensure that the fibrous constructs are covered.

FIG. 2 illustrates the collagen mesh capture frames and method. (A) Silicone gaskets and steel support frames are placed on opposite sides of a collagen microfiber mesh on the collection mandrel, the frames are collapsed together, and screws are put in place to hold the frame together. (B) Screws, frames, and gaskets used in the capture process. (C) A captured mesh in the well of a 6-well plate and ready for hydrogel/cell casting.

Calibration of the Collection Device.

Fabricated mesh fidelity relative to the Dia design file depends on the distance between rod positions stated in the Dia design file. Device calibration is necessary to ensure that the fiber angles described in the mesh protocol design step match what is produced during wet spinning and mesh fabrication. The Dia files provided with in this specification have been calibrated to a rod spacing of 1240 units (corresponding to the y-axis tick marks in the Dia software), which is appropriate for the collection device as described in this specification. Changes to the mandrel dimensions, fiber guide position, or choice of stepper motor will require adjusting or recalibration of this parameter. These steps describe the calibration process:

Using the provided Dia file (which uses a rod spacing parameter of 1240 units), a mesh is created with known target fiber angle in terms of units. The provided 30° mesh angle protocol is a good starting point.

The known mesh protocol is loaded onto the microcontroller and a set of meshes is fabricated, as described.

Each mesh on the mandrel is imaged on a microscope with a low power objective.

Fiber angle is analyzed by ImageJ using the Angle Tool. The angles of at least five overlapping fibers are measured for each mesh, and an overall empirical fiber angle mean is calculated.

A new calibration factor is determined by multiplying the ratio of the target fiber angle over the empirical fiber angle by the rod spacing parameter (initially 1240). The rod spacing parameter should increase if the empirical angle is too acute and decrease if the empirical angle is too obtuse.

Repeat steps b through d to ensure that the fiber angle is accurate.

Mechanical Analysis of Individual Collagen Fibers.

An adhesive is prepared by mixing two parts acetone and one part polyurethane adhesive (e.g., Gorilla Glue®). Thinning the polyurethane in this way decreases the viscosity so glue can be precisely placed via syringe. The prepared adhesive is loaded into a 3 mL syringe.

The chamfered tip of a 22-gauge syringe is cut, so the new opening of the needle is fully perpendicular to the length of the needle, and this needle is attached to the syringe. This step facilitates precise adhesive placement.

A printed grid of known dimensions (rectangles 14 mm×8 mm) is glued to a petri dish. Stainless steel washers (ID 1.7 mm, OD 4.0 mm, McMaster Carr) are carefully placed at each grid intersection to ensure sample regularity. Precise fiber length is measured later, immediately before testing via calipers.

Single wet spun collagen fibers are cut to approximate size and each is laid across a pair of the steel washers. An anti-static tool (such as the Milty Zerostat, or an ionizing air blower) may assist in dissipating stating electricity that can interfere with collagen fiber manipulation.

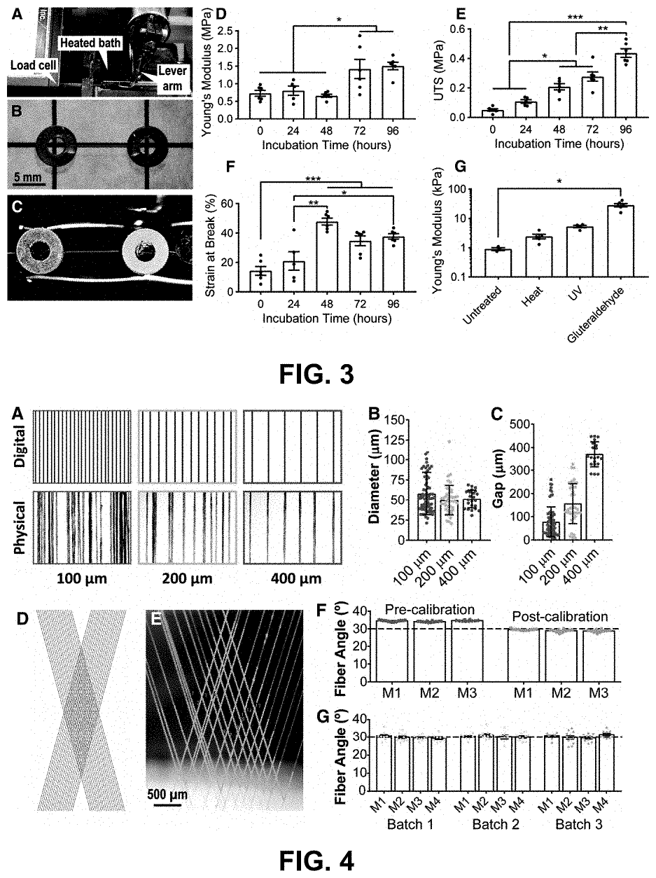

Under a dissection scope, the prepared syringe is used to deposit droplets of adhesive on the regions of fiber and washer overlap. The adhesive may dry overnight (FIG. 3B).

After the adhesive is dry, fiber samples are placed in separate wells of a six well plate, covered with four mL of phosphate-buffered saline, and incubated at 37° C. for various lengths of time before testing.

Fiber samples are carefully removed from the six well plate and are placed into the phosphate-buffered saline filled bath of a micromechanical testing apparatus (Aurora Scientific, model 801C) (see, FIGS. 3A and 3C), consisting of a temperature-controlled basin (maintained at 37° C.), a hook connected to a lever arm, and a hook connected to a five mN load cell (Aurora Scientific, model 403a). One washer is placed around each of the two hooks, a preload of 0.1 mN is applied to each sample, and the initial sample length is measured with calipers.

Each fiber sample is preconditioned before testing by pulling to 10% strain at a rate of 1% strain per second for a total of eight cycles.

After preconditioning, the 0.1 mN pre-load is re-applied, the new sample length is recorded, and each sample is pulled to break at a rate of 10% strain/min. Strain at break, ultimate tensile stress (UTS), and Young's modulus (defined based on the linear region of the stress-strain curve, typically between 10% and 30% strain) is calculated from the pull-to-break trace for each sample.

Mechanical Analysis of Fibrous Collagen Mesh Composites.

Acellular collagen mesh composites are prepared using 30° fiber meshes with 200 μm fiber spacing in stainless steel capture frames. Meshes are captured in two orthogonal orientations called longitudinal and transverse. See FIG. 5A.

Mesh composites are incubated at 37° C. in phosphate-buffered saline for six days.

Mesh composites are isolated by disassembling the stainless-steel capture frames. Aluminum T-clips (0.02 mm thick aluminum foil cut into the shape of a T, with a 0.8 mm diameter hole in the center of the longest tab) are attached to either end of the mesh composites by folding the wings inward. See FIG. 5B. This method provides a means of griping the hydrogel constructs without causing damage.

Composite samples are mounted on to a micromechanical apparatus (model 801C, Aurora Scientific) with a 37° C. bath filled with phosphate-buffered saline by positioning the two right angle hooks through the holes of the aluminum t-clips. See FIG. 3A. A preload of 0.1 mN is applied to each sample, and the initial samples length is measured with calipers.

Each composite sample is preconditioned before testing by a triangular waveform pulling to 10% strain at a rate of 1% strain per second and declining at the same rate for a total of eight cycles.

After preconditioning, the 0.1 mN pre-load is re-applied, the sample length is recorded. Each sample is pulled to break at a rate of 10% strain/min. Young's modulus (defined based on the linear region of the stress-strain curve, typically between 10% and 30% strain) is calculated from the pull-to-break trace for each sample.

Statistical Analysis.

All statistical analyses were performed in Prism 7, commercially available from GraphPad Inc., San Diego, Calif., USA. For comparison between two groups, Student's t-tests were used. For comparisons of more than two groups, one-way analysis of variance (ANOVA) with multiple comparisons and Tukey's post-hoc test was used. Error bars represent standard error of the mean unless otherwise noted. Group differences were considered statistically significant for p-values <0.05.

The following EXAMPLES are provided to illustrate the invention and should not be considered to limit its scope.

### Example 1

Single Fiber Tensile Mechanical Properties Increase with Physiological Incubation or Crosslinking.

To manipulate the mechanical properties of a composite material with embedded collagen microfibers, the mechanical properties of the fibers relative to the bulk hydrogel material are of critical importance. Collagen self-assembly could increase the stiffness and strength of the fibers in a physiological aqueous microenvironment. Individual collagen microfibers were prepared using a mesh protocol with straight fibers spaced 400 μm apart. After spinning, fibers were cut from the mandrel and glued to stainless steel washers. Samples were incubated for 0, 24, 48, 72, or 96 hours in phosphate-buffered saline at 37° C. All samples were preconditioned and then tested in a constant strain rate pull-to-break test. The zero-hour incubation group could hydrate in the testing bath for ten minutes before analysis.

Before the incubation treatment, collagen microfibers had a Young's modulus of 0.73±0.08 MPa, UTS of 0.05±0.01 MPa, and strain at break of 14.21±3.02% (FIG. 3D-F). After a 96-hour incubation, the Young's modulus increased to 1.51±0.11 MPa, UTS to 0.44±0.03 MPa, and strain at break to 37.70±1.85%, significant increases for all three metrics (p<0.05). Evaluation at time points up to 15 days showed no further significant increases vs. 96 hours.

Individual fiber mechanical samples were crosslinked either via dry heat for one hour, 254 nm, 30-Watt UV light at a distance of 75 cm for one hour, or exposure to 25% w/v glutaraldehyde vapor for twenty-four hours. Crosslinked fibers produced Young's modulus values as high as 28.1±3.5 MPa (FIG. 3G).

### Example 2

### Scanning Electron Microscopy and Transmission Electron Microscopy Imaging Show Collagen Fibril Self-Assembly

To test that collagen self-assembly increases mechanical properties with incubation in physiological solution, the inventors prepared bundles of overlapping wet spun collagen microfibers using a collection protocol with no translational movement. Bundles of twenty fibers were collected and embedded in agarose to hold fibers together during incubation and processing. One group of collagen fiber bundles was immediately stained with 3% potassium ferrocyanide, 2% osmium tetroxide and 1% uranyl acetate, embedded in EPON 812 resin, and sectioned for imaging, while a second group was incubated in a 10 cm dish filled with 10 mL of phosphate-buffered saline at 37° C. for six days before undergoing the same staining, blocking, and sectioning process.

The inventors performed electron microscopy imaging of un-incubated and incubated collagen microfiber samples to demonstrate the characteristic ability of native collagen to assemble into fibrils. Both samples were imaged by serial block face imaging (SBF), a technique that allows for layer-by-layer SEM imaging to develop a 3D visualization of the sample volume.

Serial block face imaging of unincubated fibers revealed small-diameter collagen fibrils visible along the exterior of the wet spun microfibers. Serial block face renderings of fibers incubated for 120 hours under cell culture conditions showed large, hierarchically organized collagen fibers (bundles ranging in diameter from one to 15 μm) branching out from the surface of the wet spun microfibers, primarily oriented toward the microfibers. Fiber diameters ranged from 40-100 μm dependent upon syringe pump speed during wet spinning. The inventors noted the emergence of large diameter fibrils following collagen microfiber incubation. Further transmission electron microscopy (TEM) imaging of the incubated fibers found further evidence of collagen self-assembly in clear D-banding phenomena with periodicity of 61.0±2.8 nm, in line with values found in native collagen by other groups. See, Bozec et al., Biophys. J., 92, 70 (2007) and Zeugolis et al., Biomaterials, 29, 2293 (2008). The TEM imaging results from the incubated collagen microfibers showed D-banding phenomena characteristic of native collagen, supported by D-banding measurements.

### Example 3

Differential Scanning Calorimetry Analysis Confirms Collagen Integrity after Wet Spinning

To confirm that the collagen maintained its native structure following the wet spinning process, we replicated the differential scanning calorimetry (DSC) methods of Zeugolis et al., Biomaterials, 29, 2293 (2008). Briefly, collagen microfibers were collected following the protocol described above and then both these microfibers and unprocessed, lyophilized stock collagen were incubated in phosphate-buffered saline for 24 hours at 4° C. to ensure full hydration. Samples were blotted dry on lint-free wipes and hermetically sealed in 40 μL aluminum pans. For each of the two groups, n=3 samples were sequentially heated at a constant rate of 5° C./min from 15° C. to 90° C. on a TA Instruments DSC Q20. An empty aluminum pan was used as reference. The denaturation curve of each sample was analyzed based on three metrics: onset of denaturation temperature, peak denaturation temperature, and change in enthalpy (calculated as the area under the denaturation curve).

Both the collagen microfibers and the collagen stock solution exhibited change in enthalpy and denaturation temperatures consistent with native collagen molecular structure in TABLE 2.

TABLE 2
Differential Scanning Calorimetry Analysis of
Unprocessed Collagen and Collagen Microfibers

| | ΔH_{D} | Onset | Peak |
|---|---|---|---|
| Sample | (J/q) | (° C.) | (° C.) |
| Lyophilized | −7.05 ± | 39.31 ± | 49.01 ± |
| collagen | 0.42 | 0.68 | 0.29 |
| Collagen | −5.95 ± | 41.97 ± | 51.88 ± |
| microfibers | 0.14 | 0.06 | 0.26 |

These values are distinctly greater than those associated with collagen denatured by aggressive solvents and gelatin (ΔH_{D}>−2.11, onset <37.07, and peak <40.36), suggesting that no denaturation occurred during wet spinning. The collagen wet spinning process is associated with an increase in onset and peak denaturation temperatures, as has been observed previously with extruded collagen fibers, suggesting increased molecular interactions between the triple-helical collagen molecules within wet-spun fibers.

### Example 4

### Mesh Fidelity Depends on Fiber Spacing

To evaluate the limits of fiber placement resolution, mesh protocols were prepared to deposit single, parallel fibers with spacing of 100, 200, or 400 μm. Both effective fiber spacing and fiber diameter were analyzed on the wet spinning mandrel (fiber diameter was considered because of the tendency of fibers to adhere to each other because of the accumulation of static electricity).

Results showed that at 100 and 200 μm spacing, static adhesion occurred, increasing the variability in both gap spacing and effective fiber diameter. See, FIGS. 5A and 5B. Neighboring fiber adhesion was completely eliminated with 400 μm spacing, but lower spacing values may still be desirable to achieve higher fiber densities. Effective fiber diameter remained relatively consistent across the 100, 200, and 400 μm protocols, but variability decreased when adhesion was eliminated (57.89±3.09, 49.79±2.81, and 51.26±2.28 μm, respectively).

Accuracy and precision of overlapping fiber angles were evaluated. Angled fiber placement results from several rotation steps on the stepper motor responsible for translational movement. Placement accuracy depends primarily on calibration of the software.

Conversely, calibration does not affect placement precision, and this value depends instead on the inherent variability of the collection system and its constituent components, such as the precision of the stepper motor, flexion in the Teflon and acrylic components, and unevenness in the mandrel adhesive surface. Initial fiber angle data collected in calibration (with the target of a 30° fiber angle) found a mean fiber angle of 34.4±0.1 degrees when angles between the overlapping fibers were measured by ImageJ. Following calibration, a mean fiber angle of 29.1±0.1 was achieved. See, FIG. 4E. After being properly calibrated, the collection system can produce meshes with any fiber angle, with the same degree of accuracy and precision. Repeated mesh fabrication of the same 30° fiber angle and 200 μm fiber spacing protocol demonstrated high consistency across fabrication batches. See, FIG. 4F.

### Example 5

### Mechanical Testing of Whole Meshes Demonstrates Composite Material Anisotropy

To evaluate the impact of simple anisotropic collagen fiber patterns on mechanical anisotropy, collagen microfiber meshes with 30° and 60° fiber angles were prepared as described above. Meshes were then captured in each of two orientations (diamond long axis parallel to either the long axis of the mesh window or the short axis of the mesh window. See, FIG. 5A. These meshes were embedded in 50 μL of a 2.0 mg/mL collagen solution, covered with phosphate-buffered saline and stored in culture conditions (37° C. and 5% CO₂). After six days, constructs were harvested, and aluminum t-clips were attached to each end of the construct to facilitate mechanical testing. All samples were evaluated under the same conditions as the single collagen fibers, described in FIG. 5. Young's modulus values for meshes were evaluated in both transverse and longitudinal directions (p=0.0007).

Transverse composite fiber samples with a 30° fiber angle have a Young's modulus of 4.92±1.09 kPa. The longitudinal samples have a Young's modulus of 22.28±2.50 kPa, representing a statistically significant 4.5-fold change (p=0.0007). Transverse fibers samples with a 60° fiber angle have a Young's modulus of 6.85±0.93 kPa, while longitudinal samples have a Young's modulus of 11.40±0.48 kPa, representing a statistically significant 1.7-fold change (p=0.0048).

### Example 6

### Fibrous Scaffold Composite Tissues Support Resident Cell Viability

Collagen microfiber constructs were prepared with 50 μL of a casting mix of human induced pluripotent stem cell (hiPSC)-derived cardiomyocytes (15×10⁶/mL) in 1.2 mg/mL collagen I. Constructs were cultured at 37° C., 5% CO₂ and were fed with cardiomyocyte maintenance media (RPMI+B27+Pen-Strep) every other day. After six days of culture constructs were incubated with calcein AM and ethidium homodimer-1 (Live/Dead Assay, Invitrogen) and imaged on a confocal microscope (Olympus FV3000). Replicate constructs were fixed, embedded in frozen blocks, sectioned, and stained with α-actinin, a cardiac marker, and DAPI nuclear stain.

The inventors photographed fibrous scaffold composite tissues. Gross images taken of constructs after ninety-six hours in culture demonstrate visible construct compaction, the process through which resident cells remodel the hydrogel microenvironment. Constructs compacted to 38.4%±2.77% of their initial visible area after ninety-six hours of culture (n=6) with no failure to compact and form a tissue. Fluorescent live/dead staining revealed dense, viable cardiomyocytes throughout the tissue. Immunohistochemistry confirmed the cardiomyocyte phenotype of seeded cells in the form of banded α-actinin staining of myofibril z-discs.

The inventors identified by photography that cells compact the collagen microfiber scaffold to form viable engineered tissue. The inventors observed that uncompacted 30° fiber angle construct immediately after casting and compacted 30° fiber construct after six days in cell culture conditions. Using confocal microscopy, the inventors observed an image of live/dead stained collagen microfiber construct after six days of culture. Green calcein AM was used to denote the cytoplasm of live cells, while red ethidium homodimer-1 denotes the nuclei of sparse dead cells. Fluorescent live/dead imaging revealed dense, viable cardiomyocytes throughout the tissue. Frozen block section of 30° fiber construct after six days in cell culture conditions were stained with the cardiac marker α-actinin and with DAPI. Immunohistochemistry confirmed the cardiomyocyte phenotype of seeded cells in the form of banded α-actinin staining of myofibril z-discs.

Evaluation of the stability or degradation of a new biomaterial in physiological conditions is critical to understanding material performance in biomedical applications. Individual collagen microfibers showed more robust mechanical performance with increased Young's modulus, UTS, and strain at failure, with increasing incubation time in physiological phosphate-buffered saline at 37° C. up to four days. See, FIGS. 3D-3F. After 96 hours of incubation, the Young's modulus of the non-crosslinked fibers increased to 1.51±0.11 MPa orders of magnitude greater than conventional collagen hydrogels used for tissue engineering (0.5-12 kPa). See, Kaiser et al., ACS Biomater. Sci. Eng., 5, 887 (2019) and Raub et al., Acta Biomater., 6, 4657 (2010). The Young's modulus was also greater than most native soft tissues. See, Cook & McDonagh, Eur. J. Appl. Physiol., 72, 380 (1996); Iivarinen et al., Skin Res. Technol., 20, 347 (2014); Iivarinen et al., Med. Eng. Phys., 33, 1245 (2011); and Capulli et al., Adv. Drug Deliv. Rev., 96, 83 (2016). In addition, the tensile strain at failure after 96 hours of incubation (37.70±1.85) exceeds the tensile strain regularly experienced by many native soft tissues (including cardiac muscle and tendon). See, Korinek et al., J. Am. Soc. Echocardiogr., 18, 1247 (2005) (muscle) and Maganaris & Paul, J. Physiol., 521, 307 (1999) (tendon). Together, these properties mechanically justify the utility of collagen microfibers as a reinforcing material in a composite soft tissue scaffold.

The molecular self-assembly of collagen could increase mechanical strength. Scanning electron microscopy (SEM) imaging of unincubated and 5-day incubated collagen microfibers confirmed larger collagen fibrils after incubation, particularly at the surface of the collagen microfibers. Further TEM imaging confirmed fibril self-assembly with characteristic collagen D-banding at 61.0±2.8 nm periodicity, which agrees with values reported in the literature for native collagen. See, Baselt et al., Biophys. J., 65, 2644 (1993) and Cheng et al., Biomaterials, 29, 3278 (2008). DSC evaluation of hydrated collagen microfibers demonstrated denaturation characteristics consistent with those described in the literature for triple helical collagen. Together, these findings demonstrate that the molecular integrity of the purified collagen has been maintained throughout the isolation and wet spinning process. While previous groups have reported similar findings regarding wet-spun collagen microfibers, the modifications to previously described protocols mandated by the mesh collection and embedding strategy described in this specification prescribed this validation. See, Caves et al., J. Biomed. Mater. Res. B., Appl. Biomater., 93, 24 (2010) and Pins et al., Biophys. J., 73, 2164 (1997). The molecular self-assembly that occurs within the collagen microfibers also demonstrates that (a) strength can be increased without chemical crosslinking and (b) the scaffold comprises molecular building blocks that can be utilized and remodeled by cells in tissues.

Implementation of a fiber scaffold for regenerative medicine and tissue engineering requires evaluation of the mesh itself, the acellular material, and cell viability upon contact with the biomaterial. Mesh fidelity analyses demonstrate high accuracy and precision in fiber angle, and minimal variability in fiber angle between fabrication batches. Mesh fidelity was reduced in meshes with fibers spaced less than 400 μm apart because of static attractions between the fibers during placement, causing fibers to adhere to a neighboring fiber. This level of pattern corruption may be acceptable depending on the application. Closer fiber spacing may be desirable for applications in tissue scaffolds. Scaffolds composed of fibers ˜400 μm apart would ensure that no cell in the fiber plane is more than 200 μm from a fiber with high homogeneity. Conversely, targeting 200 μm spacing with the approach described would ensure that most cells are within 100 μm of a fiber (shown to increase cellular alignment) at the cost of overall mesh uniformity. See, Chrobak et al., ACS Biomater. Sci. Eng., 3, 1394 (2017). The impact (positive or negative) of this heterogeneity will depend largely on the intended application and associated tolerances.

A major advantage of a bespoke fiber architecture is the ability to design anisotropy into the ECM scaffold, such as found in cardiac muscle, for example. Mechanical analysis performed on acellular 30° meshes captured in both longitudinal and transverse orientations demonstrated mechanical anisotropy in composite constructs evidenced by an ˜4.5:1 ratio between Young's modulus in the longitudinal orientation relative to the transverse orientation, while 60° meshes exhibited a ratio of ˜1.7:1. See,

FIG. 4. The Young's modulus of the 30° composites in the longitudinal direction (22.28±2.50) approached that of native myocardium (30.80±2.71 kPa). See, Kaiser et al., ACS Biomater. Sci. Eng., 5, 887 (2019). This fabrication method can produce materials that approach the passive stiffness of native tissues, before compaction by resident cells, which is anticipated to further increase the mechanical properties. The ratio of longitudinal to transverse Young's modulus values for the 60° composites was found to be 1.7:1, which closely approximates the range of anisotropic ratios reported in the literature for left ventricular native myocardium (˜1.4:1 to ˜2.0:1). See, Kaiser et al., ACS Biomater. Sci. Eng., 5, 887 (2019); Park et al., Biomaterials, 32, 1856 (2011); and Neal et al., Tissue Eng. Part A., 19, 793 (2013).

Many other sophisticated designs can be created for a range of applications. See, FIG. 13. Arrays of aligned collagen microfibers can be created to emulate native tendon, and nonrectangular shapes can be created to emulate both the structure and mechanics of diverse tissues, such as valve leaflets. Finally, the inventors prepared meshes with up to four layers, resulting in a hydrated mesh thickness of ˜400 μm, permitting the fabrication of organized, dense fibrous tissues that approach/exceed the diffusion limits for engineered tissues. See, Vollert et al., Tissue Eng. Part A., 20, 854 (2013); Lovett et al., Tissue Eng. Part B Rev., 15, 353 (2009); and Sarig et al., Pushing the envelope in tissue engineering: ex vivo production of thick vascularized cardiac extracellular matrix constructs. Tissue Eng. Part A., 21, 1507 (2015). See also, FIG. 13. Additional layers or modular assembly may enable thicker tissues if integrated with perfusion of nutrients in vitro or a blood supply by vasculature in vivo.

The primary advantages of this method of collagen microfiber wet spinning over other methods of fibrous scaffold production are the use of collagen I as a fiber material, the high fidelity of the meshes, and the facile process through which mesh designs can be created and fabricated. Compared to synthetic polymers, collagen I offers distinct advantages because of its high density of cell adhesion sites, tunable stiffness and degradation parameters via crosslinking and known metabolic pathways. As the primary component of most soft tissue ECMs throughout the body, collagen I requires minimal modification to satisfy a range of native-like criteria. Alternative methods of producing fibrous scaffolds with collagen may themselves damage the collagen, minimizing the benefits of using the polymer.

Despite the wide variety of scaffold technologies available to tissue engineers today, a compromise must often be made between the desirable cell adhesion and remodeling responses of natural polymers (like triple helical collagen), and the ease of production, mechanical robustness, and molecular durability of synthetic polymers, which have rapidly advanced novel fabrication technologies like 3D printing and electrospinning. Often, the solution is natural and synthetic copolymers and blends, such as polycaprolactone-collagen, collagen/polylactic acid, and collagen/poly(lactic-co-glycolic acid), to benefit from the mechanical integrity of synthetics and the cell-binding sites of collagen. See, Dippold et al., Novel approach towards aligned PCL-Collagen nanofibrous constructs from a benign solvent system. Mater. Sci. Eng. C., 72, 278 (2017); Chakrapani et al., J. Appl. Polym. Sci., 125, 3221 (2012); Haaparanta et al., J. Mater. Sci. Mater. Med., 25, 1129 (2014); and Sadeghi-Avalshahr et al., Regen Biomater 4, 309 (2017).

### Example 7

### In Vivo Data on Wet-Spun Collagen Microfibers

The inventors implanted engineered human cardiac tissues containing collagen microfibers and hiPSC-derived cardiomyocytes were implanted on the rat heart four days after ischemia/reperfusion myocardial infarction. The tissues were aligned circumferentially around the heart and held in place by sutures. The inventors observed by photography that the collagen microfibers persist for two weeks in vivo. Dense collagen microfibers were identified after two weeks in vivo by histological labeling with picrosirius red stain, which labels collagen. Morphological assessment shows round fibers of comparable diameter and staining intensity clustered together adjacent to the muscle, suggesting low to moderate swelling and remodeling after two weeks and demonstrating persistence of the uncross-linked collagen microfibers.

### List of Embodiments

Specific compositions and methods of the collagen microfiber scaffolds have been described. The detailed description in this specification is illustrative and not restrictive or exhaustive. The detailed description is not intended to limit the disclosure to the precise form disclosed. Other equivalents and modifications besides those already described are possible without departing from the inventive concepts described in this specification, as persons skilled in the tissue engineering art will recognize. When the specification or claims recite method steps or functions in an order, alternative embodiments may perform the functions in a different order or substantially concurrently. The inventive subject matter should not be restricted except in the spirit of the disclosure.

When interpreting the disclosure, all terms should be interpreted in the broadest possible manner consistent with the context. Unless otherwise defined, all technical and scientific terms used in this specification have the same meaning as commonly understood by persons of ordinary skill in the tissue engineering art to which this invention belongs. This invention is not limited to the particular methodology, protocols, reagents, and the like described in this specification and can vary in practice. The terminology used in this specification is not intended to limit the scope of the invention, which is defined solely by the claims.

All patents and publications cited throughout this specification are expressly incorporated by reference to disclose and describe the materials and methods that might be used with the technologies described in this specification. The publications discussed are provided solely for their disclosure before the filing date. They should not be construed as an admission that the inventors may not antedate such disclosure under prior invention or for any other reason. If there is an apparent discrepancy between a previous patent or publication and the description provided in this specification, the specification (including any definitions) and claims shall control. All statements as to the date or representation as to the contents of these documents are based on the information available to the applicants and constitute no admission as to the correctness of the dates or contents of these documents. The dates of publication provided in this specification may differ from the actual publication dates. If there is an apparent discrepancy between a publication date provided in this specification and the actual publication date supplied by the publisher, the actual publication date shall control.

The terms “comprises” and “comprising” should be interpreted as referring to elements, components, or steps in a non-exclusive manner, indicating that the referenced elements, components, or steps may be present, used, or combined with other elements, components, or steps. The singular terms “a,” “an,” and “the” include plural referents unless context indicates otherwise. Similarly, the word “or” should cover “and” unless the context indicates otherwise. The abbreviation “e.g.” is used to indicate a non-limiting example and is synonymous with the term “for example.”

When a range of values is provided, each intervening value, to the tenth of the unit of the lower limit, unless the context dictates otherwise, between the upper and lower limit of that range and any other stated or intervening value in that range of values.

Some embodiments of the technology described can be defined according to the following numbered paragraphs:

A composite collagen microfiber scaffold formed from wet spun collagen microfibers by coaxial flow by a buffering reaction that has native collagen molecular structure and control of microfiber diameter.

The composite collagen microfiber scaffold formed from wet spun collagen microfibers by coaxial flow by a buffering reaction that has native collagen molecular structure and control of microfiber diameter, wherein the reaction further provides control of microfiber spacing, angles, and layering.

A mesh collection and organization device with multiple scaffold windows for maintaining fiber cross-sectional shape.

A collagen fiber wet spinning and mesh device with coaxial fiber formation, a washing element (e.g., in an ethanol bath, see FIG. 1C), fiber guidance onto a collection device, and independent rotational and translational control for mesh collection.

A collagen microfiber mesh with control of microfiber spacing, angles, and layering.

An automated method of preparing collagen microfibers, comprising the steps of organizing the collagen fibers into precisely controlled mesh designs, embedding the mesh designed collagen fibers in a bulk hydrogel, and creating a composite biomaterial suitable for a wide variety of tissue engineering and regenerative medicine applications.

An automated method for the fabrication of bespoke collagen microfiber scaffolds for tissue, comprising isolating fibrillary collagen; fabricating and assembling a collection device and bath; designing microfiber mesh protocols; wet spinning collagen meshes; and capturing and embedding the wet spun collagen meshes.

The automated method for the fabrication of bespoke collagen microfiber scaffolds for tissue, further comprising the step of culturing and evaluating engineered tissues containing wet spun collagen meshes.

The automated method for the fabrication of bespoke collagen microfiber scaffolds for tissue, wherein the fibrillary collagen is vertebrate type I collagen.

The automated method for the fabrication of bespoke collagen microfiber scaffolds for tissue, wherein the fibrillary collagen is selected from the group consisting of rat tail tendon collagen, bovine dermis collagen, and human cadaver collagen.

The automated method for the fabrication of bespoke collagen microfiber scaffolds for tissue, comprising the steps of designing and calibrating the system for fabricating and assembling the device and scaffolds.

A method comprising the steps of preparing collagen microfibers, organizing the microfibers into precisely controlled mesh designs, and embedding the tissues in a bulk hydrogel.

The method, further comprising calibration and translation from digital design to physical mesh for precise control.

The method, further comprising creating a platform suitable for a wide variety of tissue engineering applications.

We claim:

1. A composite collagen microfiber scaffold formed from wet spun collagen microfibers by coaxial flow by a buffering reaction that has native collagen molecular structure and control of microfiber diameter.

2. The composite collagen microfiber scaffold, wherein the system provides control of microfiber spacing, angles, and layering.

3. A mesh collection and organization device with multiple scaffold windows for maintaining fiber cross-sectional shape.

4. A collagen fiber wet spinning and mesh device with coaxial fiber formation, a washing element, fiber guidance onto the collection device, and independent rotational and translational control for mesh collection

5. A collagen microfiber mesh with controlled microfiber spacing, angles, and layering.

6. An automated method for fabrication of bespoke collagen microfiber scaffolds for tissue comprising the steps of:
isolating fibrillary collagen;
fabricating and assembling a collection device and bath;
designing microfiber mesh protocols;
wet spinning collagen meshes;
capturing and embedding the wet spun collagen meshes; and
culturing and evaluating engineered tissues containing wet spun collagen meshes.

7. A method of preparing collagen microfibers, comprising the steps of:
organizing the microfibers into precisely controlled mesh designs; and
embedding the tissues in a bulk hydrogel.

8. The method of claim 2, further comprising:
creating a platform suitable for a wide variety of tissue engineering applications.

9. An automated method of preparing collagen microfibers, comprising the steps of:
organizing the collagen fibers into precisely controlled mesh designs,
embedding the mesh designed collagen fibers in a bulk hydrogel, and
creating a composite biomaterial suitable for a wide variety of tissue engineering and regenerative medicine applications.
